(19) United States
(12) Patent Application Publication
(10) Pub. No.: US 2005/0004165 A1
(43) Pub. Date: Jan. 6, 2005

(54) Composition and method for modulating BAR/FXR receptor activity

(76) Inventors: Barry M. Forman, Newport Beach, CA (US); Richard L. Beard, Newport Beach, CA (US); Isabelle Dussault, Thousand Oaks, CA (US); Roshantha A. Chandraratna, Laguna Hills, CA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/910,688

(22) Filed: Aug. 3, 2004

Related U.S. Application Data
(63) Continuation of application No. 10/104,385, filed on Mar. 22, 2002.
Application No. 10/104,385 is a continuation-in-part of application No. 10/006,450, filed on Nov. 19, 2001, now abandoned.

(51) Int. Cl. A61K 31/41

(52) U.S. Cl. 514/310

(57) ABSTRACT

Methods for modulating the activity of the mammalian BAR/FXR receptor. The methods include methods of treating a hypocholesterolemic mammal comprising contacting the mammal with synthetic compounds able to modulate an activity characteristic of the BAR/FXR receptor. Other methods include a method of treating colon cancer in a mammal comprising administering a compound having a BAR/FXR antagonistic activity.

### RELATED APPLICATIONS

This application is a Continuation of U.S. application Ser. No. 10/104,385, filed on Mar. 22, 2002, which is a Continuation-in-Part of U.S. application Ser. No. 10/006,450, filed on Nov. 19, 2001, now abandoned. The entire teachings of the above applications are incorporated herein by reference.

### FIELD OF THE INVENTION

The present invention is relevant to the fields of human and veterinary medicine, physiology and biochemistry, particularly in the regulation of lipid metabolism and catabolism and cholesterol synthesis and breakdown.

### BACKGROUND OF THE INVENTION

A vast array of specific metabolic, developmental, and catabolic processes appear to be directly or indirectly regulated in vivo by comparatively small molecules such as steroids, retinoids and thyroid hormones. The mechanism whereby a single such compound can contribute to the regulation of numerous different cellular events was the subject of much speculation until relatively recently, when it was discovered that these compounds each share the ability to bind to transcriptionally active proteinaceous receptors. These protein receptors, in turn, are able to bind specific cis-acting nucleic acid regulatory sequence regions, termed response elements or RE's, located upstream of the coding sequence of certain genes and to activate the transcription of these genes. Thus, the proteinaceous receptors can serve as specific, ligand-dependent regulators of gene transcription and expression.

The amino acid sequences of these various receptors were quickly found to share regions of homology, thus making each such receptor a member of a family of ligand-modulated receptor molecules. This family has been termed the steroid superfamily of nuclear hormone receptors; nuclear, because the receptors are usually found in high concentration in the nucleus of the cell.

Further study of the structural and functional relationship between the nuclear hormone receptors has shown certain characteristics in common between them in addition to sequence homology. See e.g., Evans et al. Science 240:889-895 (1988). As stated above, the nuclear hormone receptors are able to bind to cis-acting regulatory elements present in the promoters of the target genes. The glucocorticoid, estrogen, androgen, progestin, and mineralcorticoid receptors have been found to bind as homodimers to specific response elements organized as inverted repeats.

Another class of nuclear hormone'receptors, which includes the retinoid receptor RAR (retinoic acid receptor), the thyroid receptor, the vitamin D receptor, the peroxisome proliferator receptor, and the insect ecdysone receptor bind their response element as a heterodimer in conjunction with the retinoid X receptor (RXR), which in turn is positively activated by 9-cis retinoic acid. See Mangelsdorf, et al., The Retinoid Receptors in The Retinoids. Biology, Chemistry and Medicine Ch.8 (Sporn et al., eds. 2d ed., Raven Press Ltd. 1994); Nagpal and Chandraratna, Current Pharm. Design 2:295-316 (1996), which are both incorporated by reference herein. The retinoid receptors RAR and RXR, like many nuclear receptors, exist as a number of subtypes (RARα, RARβ, RARγ, and RXRα, RXRβ, and RXRγ). Additionally, each subtype may exist in different isoforms.

While the nuclear hormone receptors referenced above have all been shown to have specific ligand partners, nucleic acid and amino acid sequencing experiments and sequence alignment and comparison have revealed a class of protein molecules retaining significant sequence homology and structural similarity to the nuclear hormone receptor superfamily, but for which no corresponding ligand has yet been discovered. In fact, some of these “receptors” have been discovered to require no ligand binding to exhibit transcriptional activity. Collectively, these unassigned receptors have been collectively termed “orphan” receptors.

Products of intermediate metabolism are known transcriptional regulators in prokaryotes and lower eukaryotes such as yeast; thus there has been speculation that such metabolites may also serve this function in higher organisms, perhaps through interaction with the nuclear hormone receptors.

Farnesol is an isoprenoid involved in the mevalonate biosynthetic pathway, which leads to the synthesis of cholesterol, bile acids, porphyrin, dolichol, ubiquinone, carotenoids, retinoids, vitamin D, steroid hormones, and farnesylated proteins. Farnesyl pyrophosphate, a derivative of farnesol, is the last common intermediate in the mevalonate biosynthetic pathway.

Forman et al., Cell 81:687-693 (1995) have demonstrated that an orphan receptor, now termed farnesoid X-activated receptor (FXR) or Bile Acid Receptor (BAR), is activated by farnesol and related molecules. This reference is hereby incorporated by reference herein. BAR/FXR is expressed in the liver, gut, adrenal gland, and kidney.

The amino acid sequence of BAR/FXR reveals a conserved DNA-binding domain (DBD) and ligand-binding domain (LBD). The LBD comprises subdomains responsible for ligand binding, receptor dimerization, and transactivation. Additionally, cells expressing chimeric proteins that contain the LBD of BAR/FXR fused to the DBD of the yeast GAL4 transcription activator did not transcribe a reporter gene containing a GAL4 response element unless the BAR/FXR construct was coexpressed with another protein comprising the dimerization and ligand binding subdomains of RXR. These data suggested that BAR/FXR and RXR interact to form a transcriptionally active dimer. No interaction was seen between BAR/FXR and any other nuclear hormone receptors that were tested. Id.

Among the nuclear hormone receptors amino acid sequence homology to BAR/FXR is high in the insect ecdysone receptor (EcR), which dimerizes with an RXR homolog. When dimerized with RXRα, BAR/FXR was shown to specifically bind hsp27, an EcR response element, however, binding was not seen when BAR/FXR was expressed alone. BAR/FXR and RXR bind to certain sequences as a heterodimer.

The BAR-RXRα complex was found to be activated byjuvenile hormone III (JH III) incubation of cells transfected with RXR and BAR. The cells were also transfected with a reporter plasmid containing 5 copies of the hsp27 response element within a portion of the mouse mammary tumor virus (MTV) promoter; the promoter was positioned upstream of the firefly luciferase gene. Activation of this gene results in the expression of luciferase, which is easily quantifiable as a measure of transactivation activity. Other potential ligands, including selected steroids, and eicosanoids were found to have no effect in this system. JH III failed to activate other nuclear hormone receptors, and does not activate either BAR/FXR or RYR alone. Forman et al., Cell 81:687-693 (1995).

JH III is a derivative of farnesyl pyrophosphate. Other farnesyl derivatives have been tested for the ability to activate the BAR-RXR complex. Farnesol was demonstrated to strongly activate the heterodimer. Other derivatives such as farnesal, farnesyl acetate, farnesoic acid and geranylgeraniol activated the BAR-RXR complex somewhat less strongly; the farnesyl metabolites geraniol, squalene and cholesterol did not activate BAR-RXR. Id.

Cholesterol synthesis is closely regulated by modulation of the levels of 3-hydroxy-3-methylglutaryl-coenzyme A reductase(HMG-CoA), which regulates the conversion of 3-hydroxy-3-methylglutaryl-coenzyme A to mevalonate. Through a series of phosphorylations and a decarboxylation reaction, mevalonate is converted into 3-isopentenyl pyrophosphoric acid, which isomerizes to 3,3-dimethylallyl pyrophosphoric acid. An enzyme-mediated condensation reaction between the 5 carbon isoprenyl compounds 3-isopentenyl pyrophosphoric acid and 3,3-dimethylallyl pyrophosphoric acid results in the formation of the 10 carbon diisoprenyl compound geranyl pyrophosphoric acid. This, in turn, reacts with another molecule of 3-isopentenyl pyrophosphoric acid to form the 15 carbon compound farnesyl pyrophosphate. Two molecules of this latter compound react to form the 30 carbon molecule presqualine pyrophosphate, which is dephosphorylated to form squaline. Squaline is then cyclized to form cholesterol. Thus, HMG-CoA reductase mediates the initial formation of the isoprene units that are subsequently assembled in series and cyclized to form cholesterol.

The levels of HMG-CoA reductase are governed in part by controlling the gene transcription, translation, and by degradation of the enzyme. Farnesol has been shown to be involved in the regulation of HMG-CoA reductase degradation. Evidence exists for the synergistic promotion of HMG-CoA reductase degradation by farnesol and a sterol component, such as 25-hydroxycholesterol. See e.g., Meigs et al., J. Biol. Chem. 271:7916-7922 (1996), hereby incorporated by reference herein.

Cholesterol is the precursor of various compounds such as sterols, bile acids such as cholic acid, and the steroid hormones such as testosterone and progesterone. All these compounds retain the basic cholesterol nucleus. The more polar bile acids are formed in the liver and secreted into the small intestine, where they aid in the absorption of lipids. The formation of bile acids from cholesterol is therefore an important degradation pathway for cholesterol, and is a key determinant of the steady-state concentration of cholesterol in the body.

The rate-limiting enzyme in the formation of bile acids from cholesterol is cholesterol 7α-hydroxylase (Cyp7a). For some time it has been known that bile acids act in a negative feedback loop to limit their own production via this pathway, but the means by which this is accomplished has remained elusive. Recently, there has been evidence that Cyp7a synthesis and expression is inhibited by bile acids. Chiang, Front. Biosci. 3:D176-93 (1998) hereby incorporated by reference herein.

Despite the fact that cholesterol is essential for the synthesis of cell membranes and various hormones and other small molecules, raised levels of cholesterol, particularly in the form of low density lipoprotein (LDL), have been strongly linked to arteriosclerosis and other cardiovascular diseases. Additionally, maintenance of appropriate bile acid concentrations is important in regulating lipid metabolism, and may be useful in the prevention of colon cancer and gallstone formation.

Specifically, bile acids such as CDCA and DCA activate cyclooxygenase-2 (COX-2) transcription. COX-2 is overexpressed in many cancer cell lines and results in the production of prostaglandins capable of inhibiting apoptosis (an important element in the body's defense against cancers) and which have been implicated in the stimulation of angiogenesis and invasiveness. Inhibitors of COX-2 expression are known to decrease the size and occurrence of intestinal polyps. Thus, the maintenance of bile acid concentrations within the body may be very important.

Among currently available drugs for the treatment of hypercholesterolemia are ion exchange media such as colestipol and cholestyramine. These drugs function by sequestering bile acids in the gut; the bile acids are then excreted in the feces. Because the intestine does not reabsorb the sequestered bile acids, the bile acids are no longer available to inhibit the formation of bile acids by cholesterol degradation. As a result, bile acid synthesis is “derepressed” with the result that the steady-state concentration of cholesterol is lowered.

Unfortunately, these ion exchange drugs have been associated with an increased incidence of intestinal tumors in rodents. Additionally, since the drugs are highly charged, they are capable of adsorbing other compounds, such as ingested drugs, naturally occurring hormones, regulatory factors and the like.

A poster displayed by Neisor, Flach, Weinberger & Bentzen at an AACR conference on Nuclear Receptors in Palm Springs, Calif. held on Jan. 8-11, 1999 discussed the ability of certain 1,1-biphosphonate esters to activate BAR/FXR and to lower plasma cholesterol levels in mammals. This poster abstract is incorporated by reference herein.

A patent application was filed by certain of the present inventors and was published Dec. 21, 2000. The invention claimed in this patent application, publication number WO00/76523, was under an obligation of assignment to the same assignees as the present invention. The invention claimed therein is drawn to methods of modulating BAR/FXR receptor activity using certain compounds.

Additionally, an International Patent Application, WO00/40965 describing the role of BAR/FXR in bile acid synthesis was published on Jul. 13, 2000. No synthetic compounds for modulating cholesterol or bile acid synthesis were disclosed therein.

It is now known that BAR/FXR is integrally involved in bile acid biology. Bile acid-activated BAR/FXR induces expression of SHP (small heterodimer partner), a molecule lacking a DNA binding site which can bind many nuclear receptors. Additionally, BAR/FXR regulates the expression of ileal bile acid binding protein (IBABP), a protein which helps to prevent bile acids from exerting cytotoxic activity when they are shuttled within cells. Specific bile acid transporters are required in order for bile acids to enter cells; the expression of at least one such transporter, BSEP, is increased by bile acid-activated BAR. BAR/FXR is also involved in the bile acid mediated control of triglyceride levels; CDCA reduces triglyceride levels in humans, and BAR/FXR mediates the synthesis of CDCA. Numerous epidemiologic studies have demonstrated an association between high dietary cholesterol and the development of colon cancer. See e.g., Potter, J. D. Colorectal Cancer: Molecules And Populations J Natl Cancer Inst 91: 916-32, 1999. However, the precise mechanism by which cholesterol metabolites contribute to colon cancer remains unclear. Cholesterol is converted to bile acids in the liver which are then excreted into the gastrointestinal tract. Thus, diets high in cholesterol result in high concentrations of bile acid in intestinal contents. The link between dietary cholesterol and elevated bile acids is worrisome as fecal bile acid concentration is significantly higher in patients with colorectal cancer than in normal individuals. See Hill, M. J., Drasar, B. S., Williams, R. E., Meade, T. W., Cox, A. G., Simpson, J. E. and Morson, B. C. Faecal Bile-Acids And Clostridia In Patients With Cancer Of The Large Bowel Lancet 1: 535-9, 1975.

In addition to epidemiological studies in humans, bile acids have been shown to promote the growth of colon cancer in various rodent models. For example, oral or intrarectal administration of bile acids induced significantly greater numbers of colon adenomas and adenocarcinomas in a carcinogen-induced model of colon cancer. In humans, mutational inactivation of the APC gene initiates most colon carcinomas. Another useful rodent model of colon carcinogenesis is the APC/Min (multiple intestinal neoplasia) mouse, which develops intestinal polyps due to a mutation the APC gene. Administration of bile acids to these mice result in increased numbers of ampullary tumors. Thus, experimental results from these rodent models support the epidemiological studies linking elevated bile acid levels to an increased risk of developing colon cancer. What mechanisms underlie the link between bile acids and colon cancer? Several studies have shown that endogenous bile acids can alter the balance between cell proliferation and apoptosis. Depletion of endogenous bile acids has been reported to decrease the rate of intestinal epithelial cell proliferation in rodents Roy, C. C., Laurendeau, G., Doyon, G., Chartrand, L. and Rivest, M. R. The Effect Of Bile And Of Sodium Taurocholate On The Epithelial Cell Dynamics Of The Rat Small Intestine Proc Soc Exp Biol Med 149: 1000-4, 1975.

Conversely, acute exposure (1-2 days) to bile acids, now known to activate FXR, has been shown to lead to an increase in intestinal epithelial proliferation of as much as 3-fold. Clearly, it would not be possible to chronically sustain this acute increase in proliferation without compensatory measures that would maintain a constant epithelial cell mass. Indeed, after chronic exposure (>2-4 weeks) to bile acids, this increase in proliferation is not observed or is dramatically reduced.

In addition to cell proliferation, apoptosis is also critical to the development of colon cancer as the progression from colon adenomas to adenocarcinomas is associated with an inhibition of apoptosis. Several bile acids have been shown to induce apoptosis in colon cancer cell lines. In contrast, bile acids decreased apoptosis in a cell line derived from a benign colon adenoma. While the role of bile acids in regulating apoptosis in vivo remains to be studied, these studies suggest that bile acids may have different effects on benign adenomas as compared to the less differentiated adenocarcinomas. The precise mechanisms governing how bile acids transduce signals that ultimately regulate cell proliferation and apoptosis remain to be elucidated.

Bile acids have also been shown to regulate transcriptional events critical to colon carcinogenesis. In particular, chenodeoxycholic acid (CDCA) and DCA activate cyclooxygenase-2 (COX-2) transcription in gastrointestinal cell lines. This is significant as COX-2, which is over expressed in many colon cancers, produces prostaglandins that inhibit apoptosis and stimulate angiogenesis and invasiveness. Moreover, selective COX-2 inhibitors decrease the number and size of polyps in APC/Min mice and are currently being evaluated in clinical trials. Thus, the ability of bile acids derived from dietary cholesterol to regulate transcription may have important implications for the development of colon cancer. However, it is unknown how bile acids transduce a signal to stimulate COX-2 gene transcription.

Taken together, the above studies demonstrate a significant epidemiological and experimental association between bile acids and colon cancer. The ability of bile acids to regulate transcription, particularly combined with recent evidence that bile acids bind to BAR, indicates that BAR, as a ligand-regulated transcription factor, may mediate the activities of bile acids in colon cancer.

The identification of a nuclear receptor such as BAR/FXR as a potential mediator of colon cell growth has significant clinical value. First, this provides a molecular basis by which to refine dietary guidelines regarding cholesterol intake and the risk of developing colon cancer. Second, such findings provide a specific molecular target for future therapies aimed at treating and/or preventing colon cancer. “Hormonal” therapies could be highly effective, particularly if they are designed to be specific, relatively non-toxic (i.e. with a minimum of unwanted activities).

Thus, there remains a need in the art for methods of modulating the steady-state concentration of cholesterol and/or bile acids in vivo. Preferably such modulation would be at least somewhat selective for the bile acid synthesis pathway and would be dissociated from BAR's effect on the expression of genes not directly involved in bile acid synthesis. Additionally, it would be useful if compounds were found that selectively inhibit the expression of certain BAR/FXR while not inhibiting, or actually increasing, the expression of other BAR/FXR target genes.

### BRIEF DESCRIPTION OF THE DRAWINGS

FIG. 1A Shows the activation of CV-1 cells transfected with CMX-BAR, CMX-RXR and the EcRE×6 TK luc reporter. Reporter activation was measured in transfectant cells alone and upon treatment with 100 μM CDCA, 5 μM AGN 29, 5 μM AGN 31, 5 μM TTNPB and 100 nM LG268.

FIG. 1B shows the activation of CV-1 cells transfected with CMX-BAR, CMX-RXR and the EcRE×6 TK luc reporter. Cells were also transfected with the RAR fusion vector Gal-L-RAR. Reporter activation was measured in transfectant cells alone and upon treatment with 100 μM CDCA, 5 μM AGN 29, 5 μM AGN 31, 5 μM TTNPB and 100 nM LG268.

FIG. 1C shows the activation of CV-1 cells transfected with CMX-BAR, CMX-RXR and the EcRE×6 TK luc reporter. Cells were also transfected with the RXRα fusion vector Gal-L-RXR. Reporter activation was measured in transfectant cells alone and upon treatment with 100 μM CDCA, 5 μM AGN 29, 5 μM AGN 31, 5 μM TTNPB and 100 nM LG268.

FIG. 2A shows the activation of CV-1 cells transfected with full length BAR/FXR upon treatment with increasing doses of CDCA, AGN 29 and AGN 31.

FIG. 2B shows the change in polyacrylamide gel electrophoresis migration of BAR:RXRm heterodimers upon incubation with the receptor interaction domain of the co-activator GRIP 1 and differing amounts of either AGN 29 or AGN 31.

FIG. 3A shows a Northern blot analysis of IBABP expression in Caco-2 cells upon incubation alone or in the presence of CCDA, AGN 29, or AGN 31.

FIG. 3B shows a Northern blot analysis of CYP7A, SHP and GAPDH expression in HepG2 cells upon incubation alone or in the presence of CCDA, AGN 29, or AGN 31.

FIG. 4A shows the results of a co-transfection assay conducted in which the combined effects of AGN 34 and CDCA on Bar/FXR activity were determined.

FIG. 4B shows a dose-response curve of transactivation activity whe3n cells are incubated in the presence of a constant amount of CDCA and increasing amounts of AGN 34.

FIG. 4C shows the effect upon the transactivational activity of a variety of nuclear receptors of incubation with AGN 34.

FIG. 4D shows a “gel shift” co-activator (GRIP) recruitment assay of AGN 34 and AGN 29, alone and together, in which the concentration of AGN 34 is increased.

FIG. 5A is a Western blot of IBABP, SHP and GAPDH RNA expression upon treatment of Caco-2 cells with CDCA, AGN34 and CDCA and AGN 34.

FIG. 5B is a Western blot of IBABP, SHP and GAPDH RNA expression upon treatment of HepG2 cells with CDCA, AGN34 and CDCA and AGN 34.

### SUMMARY OF THE INVENTION

The present invention is directed to methods for modulating the transcriptional activity of BAR/FXR through the use of synthetic ligands of the BAR:RXR heterodimer. Such ligands are able to cause BAR, preferably in combination with another nuclear hormone receptor such as RXR, to suppress, inhibit, or stimulate the transcription of a given target gene. In one preferred embodiment, the invention is directed to methods for stimulating BAR/FXR activity comprising administering an effective dose of a synthetic agonist of BAR/FXR activity. Preferred synthetic agonists are identified as AGN 29 and AGN31.

In another preferred embodiment the invention is directed to methods for inhibiting the BAR-mediated stimulation of Intestinal Bile Acid Binding Protein (IBABP) gene expression, comprising administering an effective dose of AGN 34.

Contemplated by the present invention are methods for regulating the concentration of bile acids in a mammal. A heightened concentration of bile acids in mammals has been associated with an increased occurrence of colon cancer; thus, the use of BAR/FXR ligands which do not significantly increase, or which decrease Cyp7a expression may effectively lower abnormally high bile acid concentrations therefore providing a therapeutic and/or prophylactic effect for this indication. The transcription of proteins other than Cyp7a are regulated by bile acids; these include Intestinal Bile Acid Binding Protein and Cyclooxygenase 2 (both up-regulated by CDCA), and sterol-27-hydroxylase, Intestinal Bile Acid Transporter, and Liver Bile Acid Transporter (these proteins are down regulated by CDCA). The methods of the present invention are therefore useful in modulating the expression of these proteins as well.

Thus, in another preferred embodiment the present invention is directed to a method of treating colorectal cancer through the administration to a patient in need thereof of a pharmaceutically effective dose of a BAR/FXR ligand which causes a decrease or inhibition of the formation of a IBABP:bile acid complex. Preferably the BAR/FXR ligand decreases the expression of IBABP without antagonizing at least one other activity characteristic of agonism of the BAR/FXR receptor. Particularly preferred as a BAR/FXR ligand is AGN 34.

By “BAR/FXR ligand” is meant that the ligand binds either to the BAR/FXR receptor or to a complex intermolecular complex or multimer which comprises the BAR/FXR receptor and which modulates an activity associated with the BAR/FXR receptor.

The BAR/FXR ligands of the present invention may be BAR/FXR antagonists, BAR/FXR inverse agonists, or have attributes of more than one of these. By “agonist” is meant that the ligand stimulates a ligand-dependent BAR/FXR activity above any baseline levels present in the absence of ligand. By “antagonist” is meant that the ligand binds to BAR, and functions as a competitive or non-competitive inhibitor of BAR/FXR agonist activity. By “inverse agonist” is meant that the ligand will bind to BAR/FXR and cause the suppression of an BAR/FXR activity to a level lower than seen in the absence of any BAR/FXR ligand.

By modulating an activity associated with the BAR/FXR receptor is meant that the ligand affects an activity associated primarily with the BAR/FXR receptor alone or in combination with another factor, with the BAR:RXR heterodimer, but not with the RXR homodimer. A ligand may exert its activity by binding the BAR/FXR subunit, by binding the RXR subunit, or by binding both the BAR/FXR and RXR subunit. The mechanism of modulation is irrelevant to this invention.

In another aspect the present invention pertains to methods of stimulating or inhibiting an activity, or stimulating one activity and inhibiting another activity associated with a BAR/FXR receptor of a mammal by treating such a mammal with a pharmaceutically acceptable composition comprising a compound selected from the group consisting of: AGN 29, AGN 31 and AGN 34.

AGN 29 has the following structure:

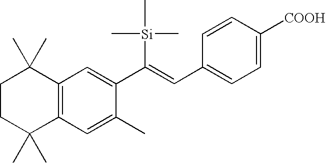

AGN 31 has the following structure:

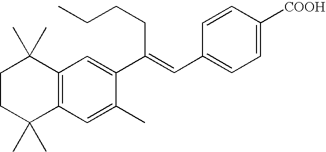

AGN 34 has the following structure:

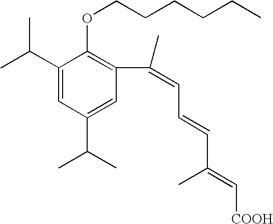

Other aspects and embodiments of the invention are contained in the disclosure that follows and the claims that conclude this specification.

### DETAILED DESCRIPTION OF THE INVENTION

The present invention is directed to methods for modulating the activity of a mammalian BAR/FXR receptor, preferably the human BARIFXR protein.

Such methods involve the use of a BAR/FXR ligand which will bind the BAR/FXR receptor or a complex containing the BAR/FXR receptor, thereby affecting the ability of BAR/FXR to exert its biological effects, either directly or by blocking the ability of a naturally occurring ligand to exert its affects. The BAR/FXR ligands of the present invention may be BAR/FXR antagonists, BAR/FXR agonists, or BAR/FXR inverse agonists. Preferably, although not necessarily, the BAR/FXR ligands have substantially no activity at the retinoid nuclear receptors, RAR and RXR. In another embodiment, the BAR/FXR ligand may be a bi-specific compound able to bind and modulate both RXR and BAR.

Also included are aspects of the invention directed to methods for increasing the plasma concentration of cholesterol in a mammal pathologically deficient in cholesterol through the use of an BAR/FXR agonist.

While not wishing to be bound by theory, the Applicants currently believe that the BAR/FXR receptor, when bound by an BAR/FXR agonist, may inhibit the transcription of the oxysterol receptor LXRα, which in turn activates transcription of Cyp7a. Repression of transcription of this key enzyme in the biosynthesis of bile acids therefore results in a lower concentration of bile acids within the body; high bile acid concentrations have been associated with a heightened risk of colon cancer.

As an aid in the further understanding of this invention, Applicants offer the following Examples, which are intended to illustrate the invention, but not to limit the scope of the claims.

Materials and Methods

All mammalian expression vectors were derivatives of the bacterial/mammalian shuttle vector pCMX, an expression vector containing the cytomegalovirus (CMV) promoter/enhancer, followed by a bacteriophage T7 promoter for transcription of the cloned gene in vitro. Plasmid pCMX also contains the SV40 small t intron/poly adenylation signal sequence, polyoma virus enhancer/origin and the SV40 enhancer/origin of plasmid CDM8 (see Seed, Nature 329:840-842 (1987), hereby incorporated by reference herein) cloned into the large Pvu II fragment of pUC19. PUC19 is a commonly used cloning vector available from New England Biolabs, Inc. This Pvu II fragment contains a Col El origin of replication and an ampicillin resistance gene for plasmid selection, but lacks the pUC 19 polylinker cloning site. To create a new polylinker site, a synthetic polylinker comprising the following restriction sites: 5′-KpnI, EcoRV, BamHI, MscI, NheI-3′ followed by a translational termination sequence inserted in all three reading frames. See Umesono et al., Cell 65:1255-1266 (1991), hereby incorporated by reference herein.

The nucleic acid regions encoding the following full-length proteins were cloned into a CMV expression vector. The sequences of these genes and/or their corresponding polypeptides have the indicated GenBank accession numbers: rat BAR/FXR (accession # U18374), human RXRα (accession # X52773), human TRβ (accession # X04707), human LXRα (accession # U22662), mouse CARβ (accession # AF009327), mouse PPARα (accession # X57638), mouse PPARδ (accession # U10375), mouse PPARγ(accession # U10374), VDR (accession # NM_{—}000376). Gal4 fusions containing the indicated protein fragments were fused to the C-terminal end of the yeast Gal4 DNA binding domain (amino acids 1-147, accession # X85976): Gal-RAR (human RAR ligand binding domain, Glu 156-Ser 463, accession # X06614), Gal-RXR (human RXRα 1gand binding domain, Glu 203-Thr 462, accession # X52773). The βgal contains the E. coli β-galactosidase coding sequences derived from pCH110 (accession # U02445). RXRm contains a single point mutation (Asp-322-Pro) in the LBD of human RXRα. Luciferase reporter constructs (TK-luc) contain the Herpes virus thymidine kinase promoter (−105/+51) linked to the indicated number of copies of the following response elements: hsp27 EcRE×6 (see Yao et al., 71 Cell 63-72 (1992)); IBABP IR-1×3 (CCTTAAGGTGAATAACCTTGGGGCTCC) (SEQ ID NO: 13); UAS_{G}×4 (MH100×4); PPRE×3 (see Forman et al., 81 Cell 687 (1995)); βRE2×3 (see Forman et al., 395 Nature 612 (1998); LXRE×3 (see Willy et al., 9 Genes Dev. 1033 (1995)); SPP×3 (see Umesono et al, 65 Cell 1255 (1991)) and T₃RE (MLV)×3 (see Perlmann et al., 7 Genes Dev. 1411 (1993). All references, including each of those above, cited in this application are hereby incorporated by reference herein unless specifically indicated otherwise. The GenBank information corresponding to these accession numbers is hereby incorporated by reference herein in its entirety. The rat BAR/FXR amino acid sequence, mouse BAR/FXR amino acid sequence, and human BAR/FXR amino acid sequence are provided herein as SEQ ID NO: 1, SEQ ID NO: 2, and SEQ ID NO: 3, respectively. The human RXRα amino acid sequence is provided herein as SEQ ID NO: 4.

GAL4 fusion proteins were constructed using standard molecular biological methods (see e.g., Sambrook et al., Molecular Cloning: A Laboratory Manual (2d ed. Cold Spring Harbor Laboratory Press 1989), incorporated by reference herein in its entirety) by inserting a nucleotide sequence encoding the indicated polypeptide immediately downstream of the yeast GAL4 DNA-binding domain in plasmid pSG424, described in Sadowski et al., Nucleic Acids Research 17:7539, hereby incorporated by reference herein. The amino acid sequence of the yeast GAL4 DBD, hereby designated SEQ ID NO: 5, is as follows:

NH2-MKLLSSIEQA CDICRLKKLK CSKEKPKCAK CLKNNWECRY
SPKTKRSPLT RAHLTEVESR LERLEQLFLL IFPREDLDM
ILKMDSLQD IKALLTGLF VQDNVNKDAV TDRLASVETD
MPLTLRQHRI SATSSSEESS NKGQRQLTVS-COOH

Fusion proteins were made, as indicated above, using common molecular biological techniques by creation of open nucleic acid reading frames encoding the indicated polypeptides, and cloning into the polylinker portion of pCMX.

For GAL-L-RXR, the plasmid nucleic acids encoded amino acids Glu₂₀₃ to Thr₄₆₂ of human RXRα (SEQ ID NO: 4) fused to the GAL4 sequences. The junction between the carboxyl terminal section of GAL4 and the amino terminal portion of the RXR LBD had the following structure:

```
                                      EcoRI Asp718 Sal/Xho
GTA-TCG-CCG-GAA-TTC-GGT-ACC-GTC-GAG-GCC-GTG-CAG-GAG-..
Val-Ser Glu-Ala-Val-Gln-Glu-..
    GAL4 -> 203 --> hRXRa LBD
```

This junction nucleotide sequence

5′GTATCGCCGGAATTCGGTACCGTCGAGGCCGTGCAGGAG3′

is hereby designated SEQ ID NO: 6.

For GAL-L-BAR, the plasmid nucleic acids encoded amino acids Leu₁₈₁ to Gln₄₆₉ of rat BAR/FXR (SEQ ID NO: 1) fused to the GAL4 sequences. The junction between the carboxyl terminal section of GAL4 and the amino terminal portion of the BAR/FXR LBD had the following structure:

```
EcoRI former
                    | KpnI/NaeI
  GTATCGCCGGAATTCGGGCTAAGGAAGTGCAGAGAGATGGGAATGTTGGCTGAATG
  ValSerProGluPheGlyLeuArgLysCysArgGluMetGlyMetLeuAlaGlu
  GAL4>| |<---rBARa AA 181 |<---LBD
```

This junction nucleotide sequence (from 5′ to 3′)

GTATCGCCGGAATTCGGGCTAAGGAAGTGCAGAGAGATGGGAATGTTG
GCTGAATG

is hereby designated SEQ ID NO: 7.

RXR ligand binding domain (LBD) expression construct L-RXR contains nucleotide residues encoding the SV40 Tag nuclear localization signal sequence (from amino to carboxy ends:

| | |
|---|---|
| APKKKRKVG | (SEQ ID NO: 8) |

located immediately upstream (i.e., to the 5′ side on the coding strand) of a nucleotide sequence encoding the human RXRα LBD (Glu₂₀₃ to Thr₄₆₂). CMX-βgal contains the E. coli β-galactosidase coding sequence derived from plasmid pCH110 (accession number U 02445) inserted downstream of the CMV promoter in plasmid pCMX. RXRm contains a single point mutation changing Asp-322 to Pro in the LBD of human RXRα.

Luciferase reporter plasmids (termed TK-Luc) were constructed by placing the cDNA encoding firefly luciferase immediately downstream from the herpes virus thymidine kinase promoter (located at nucleotide residues −105 to +51) of the thymidine kinase nucleotide sequence), which is linked in turn to the various response elements. The promoter region of the TK-Luc plasmids has the following structure:

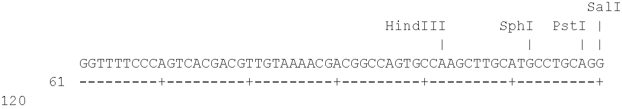

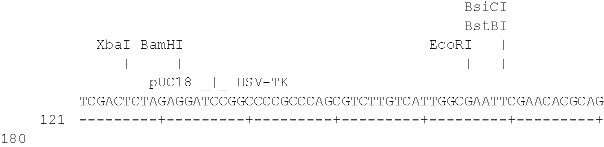

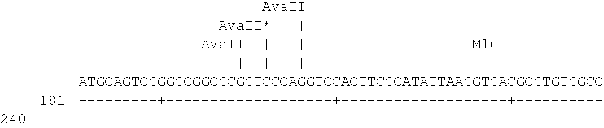

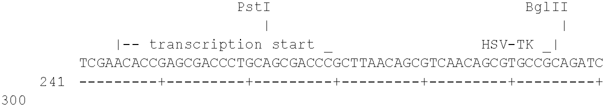

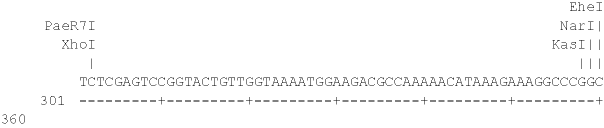

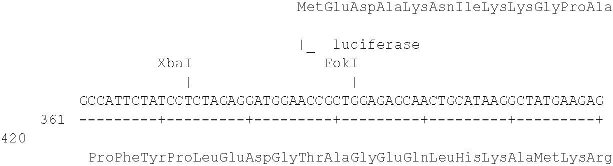

This nucleotide sequence (continuous from 5′ to 3′)

5′-GGTTTTCCCAGTCACGACGTTGTAAAACGACGGCCAGTGCCAAGCTT
GCATGCCTGCAGGTCGACTCTAGAGGATCCGGCCCCGCCCAGCGTCTTGT
CATTGGCGAATTCGAACACGCAGATGCAGTCGGGGCGGCGCGGTCCCAGG
TCCACTTCGCATATTAAGGTGACGCGTGTGGCCTCGAACACCGAGCGACC
CTGCAGCGACCCGCTTAACAGCGTCAACAGCGTGCCGCAGATCTCTCGAG
TCCGGTACTGTTGGTAAAATGGAAGACGCCAAAAACATAAAGAAAGGCCC
GGCGCCATTCTATCCTCTAGAGGATGGAACCGCTGGAGAGCAACTGCATA
AGGCTATGAAGAG-3′

is designated SEQ ID NO: 9.

Response elements were inserted in plasmid TK-Luc at the unique Hind III site. As anr example, the yeast GAL4 UAS_{G} response element has the nucleotide sequence, and was inserted in 4 direct repeats to yield UAS_{G}×4:

| | |
|---|---|
| 5′-CGACGGAGTACTGTCCTCCGAGCT-3′ | (SEQ ID NO: 10) |

As another example, the hsp EcRE (ecdysone response element) was inserted into the Hind III site of plasmid TK-Luc as six direct repeats of the following sequence:

| | |
|---|---|
| 5′-TGGACAAGTGCATTGAACCCTT-3′ | (SEQ ID NO: 11) |

to yield hsp EcRE×6.

The person of ordinary skill in the art will recognize that the sequences of other response elements discussed herein are disclosed in the cited references, and are readily available e.g., from the National Institutes of Health's National Center for Biotechnology Information (NCBI) website (http://www.ncbi.nlm.nih.gov/) on the Worldwide Web, which is hereby incorporated by reference herein in its entirety.

Transient Transfection Assay

CV-1 African Green Monkey cells were grown in Dulbecco's Modified Eagle's medium supplemented with 10% resin-charcoal stripped fetal bovine serum (FBS), 50 U/ml penicillin G and 50 μg/ml streptomycin sulfate (DMEM-FBS) at 37° C. in 5% CO₂. One day prior to transfection, cells were plated to 50-80% confluence using phenol red-free DMEM-FBS. Cells were transiently transfected by lipofection as described in Forman et al., 81 Cell 687 (1995). Liposomes (N-[1-(2,3-dioleoyloxy)propyl]-N,N,N-ammonium methyl sulfate, sold by Boehringer Mannheim under the name DOTAP) were formed according to the manufacturer's instructions. The liposomes contained reporter gene constructs (300 ng/10⁵ cells); cytomegalovirus driven expression vectors (25 ng/10⁵ cells) were added as indicated along with CMX-βgal (500 ng/10⁵ cells) as an internal control. After 2 hours the liposomes were removed and replaced with fresh media. Cells were incubated for approximately 40 hours with phenol red-free DMEM-FBS containing the indicated compounds. After exposure to the specified ligand, the cells were harvested.

The harvested cells were assayed for the presence of luciferase activity. Cells were lysed in 0.1 M KPO₄ (pH 7.8), 1.0% TRITON® X-100, 1.0 mM dithiolthreitol (DTT) and 2 mM ethylenediamine tetracetic acid (EDTA). Luciferase activity was measured by reaction of the cell lysates with luciferin in a reaction buffer comprising: 20 mM tricine, 1.07 mM Mg(CO₃)₄-Mg(OH)₂-5 H₂0, 2.67 mM MgSO₄-7H₂0, 0.1 mM EDTA, 0.5 mM Sodium luciferin, 0.15 mg/ml Coenzyme A, 5 mM DTT, and 0.5 mM adenosine triphosphate (ATP). Resulting chemiluminescence was measured in a luminometer. See de Wet et al., Mol. Cell Biol. 7:725 (1987) (hereby incorporated by reference herein). All points were assayed in triplicate and varied by less than 15%. Each experiment was repeated three or more times with similar results. No cytotoxicity was observed with any of the compounds when used at the indicated concentrations and treatment times.

Northern Analysis

HepG2 cells (a hepatoma cell line used as a hepatocyte model) were maintained in Eagle's minimal essential medium supplemented with 10% FBS, 1 mM sodium pyruvate, 2 mM L-glutamine, non-essential amino acids, 50 U/ml penicillin G and 50 μg/ml streptomycin sulfate. Caco-2 cells (a cell line derived from a colon carcinoma capable of spontaneously differentiating into cells sharing characteristics with small intestinal cells) were maintained in DMEM supplemented with 20% FBS, 50 U/ml penicillin G and 50 μg/ml streptomycin sulfate. Caco-2 cells were maintained for 20 days post-confluence to allow differentiation. They were fed twice a week with their regular media during this period.

One day prior to treatment, confluent HepG2 and differentiated Caco-2 cells were switched to phenol red-free media containing resin-charcoal stripped FBS and then treated for and additional 24 hours with the indicated compounds. Total RNA was isolated using the Trizol reagent. Northern blots were prepared from polyA⁺ RNA using the Oligotex method (Qiagen) and analyzed with the following probes: human CYP7A (accession # M93133) nucleotides 1617-2576 (Karam and Chiang, 185 Biochem. Biophys. Res. Commun. 588 (1992)), human SHP (accession # L76571) nucleotides 888-1355, human IBABP (accession # AI311734), an EST containing the entire coding sequence of IBABP.

Coactivator Recruitment Assay

Coactivators, which bind agonist-activated nuclear receptors including, without limitation, RAR and RXR, function to assist the activated nuclear receptor exert its activity as a transcription factor. Characterized co-activators include CBP, p300, RIP140, SRC-1, ACTR, TIF2 (also called GRIP 1) and TIF1. In most cases coactivator will not bind the receptor in the absence of an agonist, moreover, receptor antagonists will often block coactivator binding, either directly or through the recruitment of a corepressor. Coactivator recruitment assays are therefore a valuable method for directly visualizing associative changes to a receptor caused by the addition of a prospective ligand.

GRIP 1 was expressed as a fusion protein with glutathione-S-transferase, an enzyme which selectively binds glutathione and can thus be used as an affinity reagent. See e.g., U.S. Pat. No. 5,654,176. The GST-GRIP1 fusion protein was expressed in E. coli and purified on glutathione-Sepharose columns. In vitro translated BAR/FXR/RXR (0.6-1.2 μl each) and GST-GRIP 1 (5 μg) were incubated for 30 min at room temperature with 100,000 cpm of the E. coli DNA polymerase Klenow fragment-labeled probes in 10 mM Tris (pH 8.0), 50 mM KCl, 6% glycerol, 0.05% NP-40, 1 mM DTT, 12.5 ng/μl poly dI-dC, and the indicated ligands. Complexes were electrophoresed through 5% polyacrylamide gel in 0.5% TBE (45 mM Tris-base, 45 mM boric acid, 1 mM EDTA) at room temperature. Oligonucleotide probes were made to identify specific receptor dimers; an optimized DR-1 (direct repeat 1; a nucleotide sequence motif to which RXR will bind) probe (5′-GCTACCAGGTCAAAGGTCACGTAGCT) (SEQ ID NO: 12) was used for RXR homodimers and the IR-1 sequence of the IBABP promoter, to which the BAR/RXR heterodimer binds, was used for BAR/RXR heterodimers.

### EXAMPLE 1

### Identification of Synthetic BAR/FXR Azonists

Because of the central role played by BAR/FXR in cholesterol degradation/bile acid formation, we proposed that effective BAR/FXR activators and repressors could have beneficial pharmacological effects in a variety of bile acid-related diseases. As our starting point in the identification of BAR/FXR modulators, we made use of the observation that the synthetic retinoid TTNPB, [E]-4-[2-(5,6,7,8-tetrahydro-5,5,8,8-tetramethyl-2-naphthalenyl) propen-1-yl]benzoic acid, activates BAR. TTNPB has the structure:

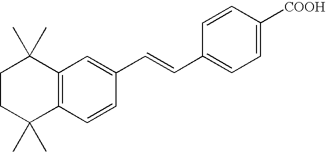

We used a sensitive, high throughput, cell-based transient transfection assay, described above, to screen for BAR/FXR activators. Using this method, we identified 2 potent agonists which we designated AGN29 and AGN31. Full-length BAR/FXR and RXR expression plasmids were cotransfected in CV-1 cells with a luciferase reporter construct containing BAR/FXR binding sites derived from the IBABP promoter (IBABP IR-1).

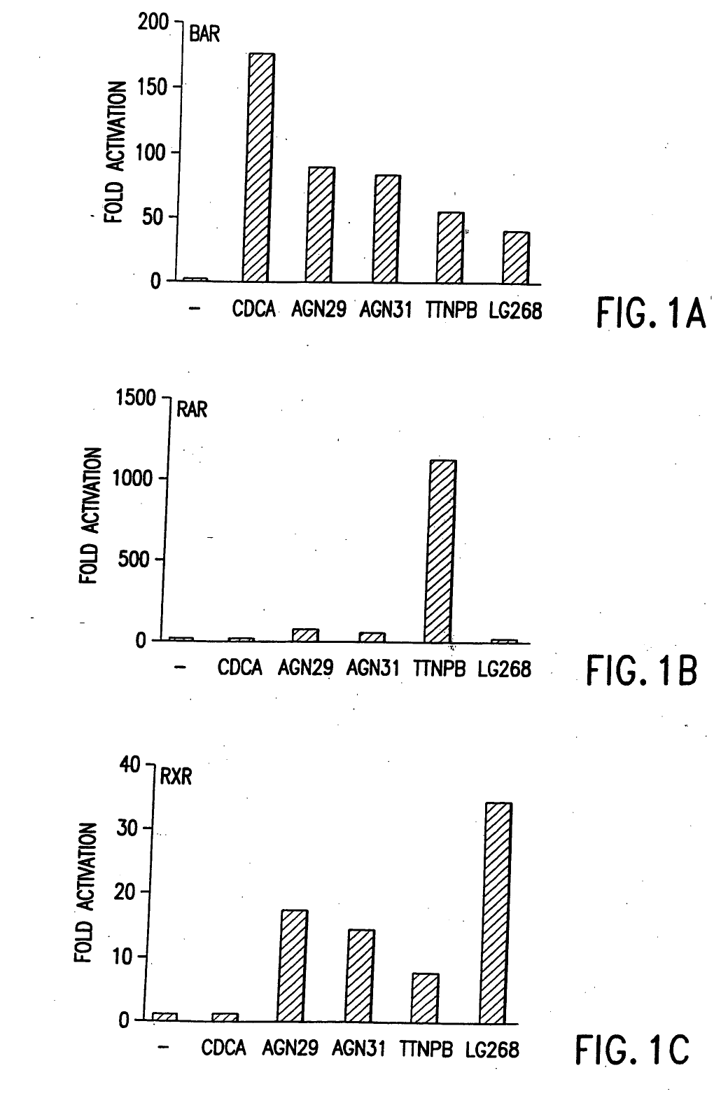

The transfected cells were then treated with the indicated ligands for 40 hours. Activity was measured using luciferase expression in the cotransfection assay described above. AGN 29 and AGN 31 robustly activated BAR/FXR (91- and 85-fold respectively) when used at a concentration of 5 μM, whereas CDCA resulted in a nearly 200-fold activation at 100 μM (FIG. 1A). TTNPB, as previously demonstrated, also induced significant BAR/FXR activity (65-fold). The RXR-specific ligand LG268 also activated the BAR-RXR heterodimer via the RXR subunit as previously demonstrated (FIG. 1A). Since TTNPB is a strong RAR activator, we tested the ability of AGN 29 and AGN 31 to activate both RAR and RXR. To eliminate the effect of ligands on endogenous receptors from our assay, we used fusion proteins between the Gal 4 DNA binding domain (DBD) and the ligand binding domain (LBD) of the receptor of interest. Thus, Gal-L-RAR or Gal-L-RXR fusions were transfected into CV-1 cells with a Gal 4 reporter (MH100×4) and the effect of different ligands was evaluated. As seen in FIG. 1B, TTNPB was able to strongly activate RAR, but treatment with AGN 29 and AGN 31 resulted in a loss of specificity for RAR. These compounds on the other hand retained some ability to activate RXR (FIG. 1C), but had no effect on other RXR heterodimers containing nuclear receptors including AR, mPXR, hPXR, ERα, CARβ, LXRα, PPARα, PPARγ, PPARδ, VDR and TRβ.

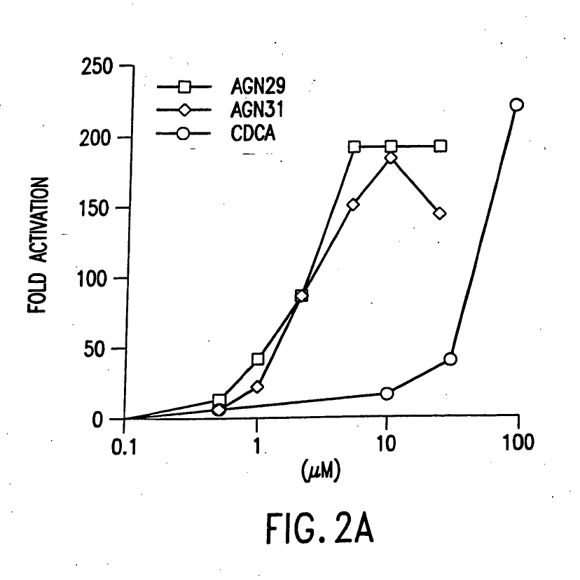

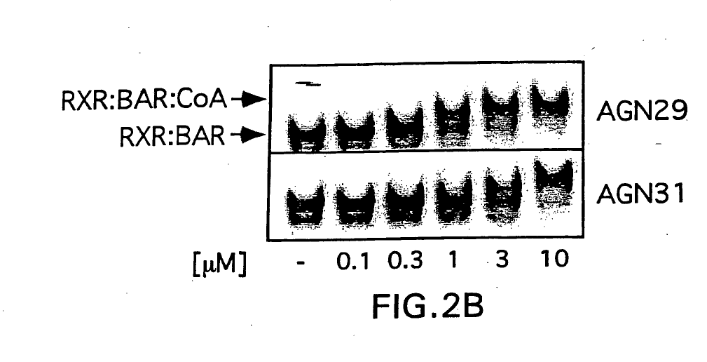

To test the relative potency of the compounds, CV-1 cells transfected with full length BAR/FXR were treated with increasing doses of AGN 29 and AGN 31. As shown in FIG. 2A, the EC₅₀ for AGN29 and AGN31 is approximately 2 μM compared with an EC₅₀ of approximately 50 μM for CDCA. By EC₅₀ is meant the concentration at which the response is 50% of maximal.

To address the question of whether the two identified compounds are ligands for BAR, we used an in vitro co-activator recruitment assay. Most nuclear receptors, including BAR, are inactive in the absence of an agonist because they are unable to interact with co-activators. In the presence of agonist, LBD appears to undergo conformational changes that enable recruitment of co-activators and subsequent transcription. By recruitment is meant the formation of a reversible non-covalent association between a receptor LBD and a co-activator or co-repressor, usually in response to the addition of such a receptor co-modulator. Because the association between co-activator and receptor results in the formation of a new higher molecular weight species, the ligand-dependent recruitment of co-activator can be detected in gel shift assays and is commonly used as an indicator for ligand binding. Blumberg et al., 12 Genes Dev. 1269 (1998); Forman et al., 395 Nature 612 (1998); Kliewer et al., 92 Cell 73 (1998); Krey et al., 11 Mol. Endocrinol. 779 (1997).

Co-activator recruitment assays were performed by mixing AGN 29 and AGN 31 with BAR, RXRm, a ³²P-labeled BAR/FXR response element (IBABP IR1) and the receptor interaction domain of GRIP 1. RXRm is an RXR mutant impaired in its ability to bind ligand and used to determine whether AGN 29 and AGN 31 bind to the BAR/FXR subunit and not to RXR. After the binding reactions, the resulting complexes were separated on polyacrylamide gels. As expected, BAR-RXRm heterodimers failed to recruit co-activator in the absence of ligand (FIG. 2B). However, addition of AGN 29 or AGN 31 shifted the majority of the complexes with the co-activator GRIP 1 suggesting that AGN 29 and AGN 31 bind BAR/FXR and not RXR. These experiments were performed with a range of doses for AGN 29 and AGN 31 and co-activator recruitment was seen at doses that parallel the potency of these compounds in transient transfection assays (FIG. 2B).

### EXAMPLE 2

We next tested the ability of AGN 29 and AGN 31 to regulate BAR/FXR target genes. We used the hepatoma cell line HepG2 as a hepatocyte model as this cell line has been used extensively to study cholesterol metabolism and bile acid-mediated gene regulation. We monitored the expression of two well known BAR/FXR target genes: CYP7A, the rate-limiting enzyme for bile acid synthesis, is down regulated by bile acids in HepG2 cells. It is now clear from gene-targeting studies that bile acids exert their inhibitory role on this gene through BAR/FXR (Sinal et al., 102 Cell 731 (2000). We also monitored the expression of the nuclear receptor SHP, a recently recognized target gene for BAR. Caco-2 cells were used as a model for ileal enterocytes. This cell line was used to monitor the levels of SHP and IBABP. Both cell lines were treated for 24 hours with AGN 29 or AGN 31 (10 μM), or with CDCA (100 μM) as a positive control.

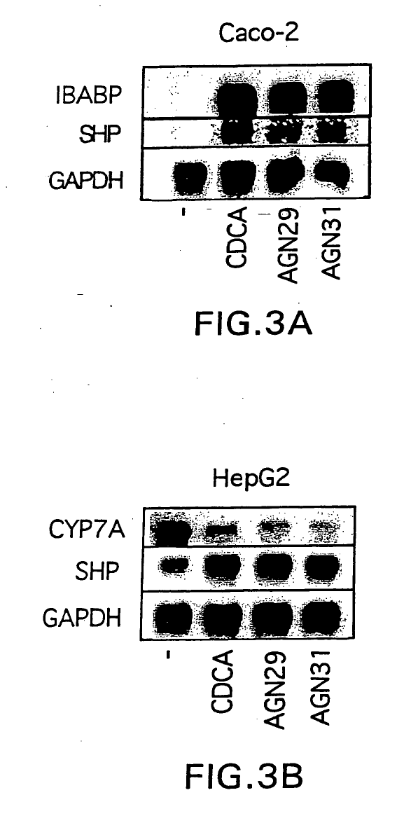

As assayed by Northern blot analysis, in differentiated Caco-2 cells AGN 29 and AGN 31 strongly induced IBABP and SHP expression to the same level as CDCA (FIG. 3A). In HepG2 cells, AGN29 and AGN31 were able to repress CYP7A expression and induce the transcription of SHP (FIG. 3B). These ligands had no effect on BAR/FXR expression or on GAPDH These results complement the in vitro data and confirm that AGN 29 and AGN 31 are agonist ligands for BAR.

### EXAMPLE 3

We next asked whether compounds previously identified by us in these assays as incapable of activating BAR/FXR might possess antagonist activity. We identified one such compound, AGN34, whose structure is shown above. To test the ability of AGN34 to repress BAR/FXR activity, we transiently transfected CV-1 cells with full length BAR/FXR and RXR and with the IR-1-containing luciferase reporter gene. Cells were then treated with suboptimal levels of CDCA (50 μM) with or without AGN 34 (1 μM). Addition of 50 μM CDCA resulted in nearly 100-fold activation of BAR.

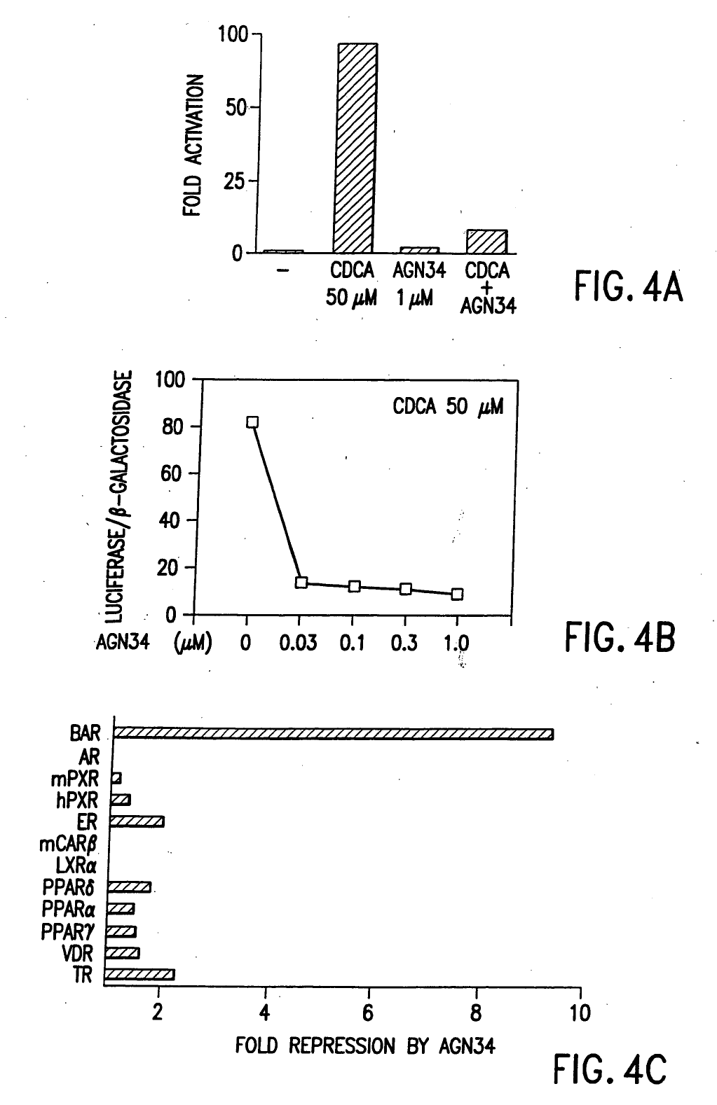

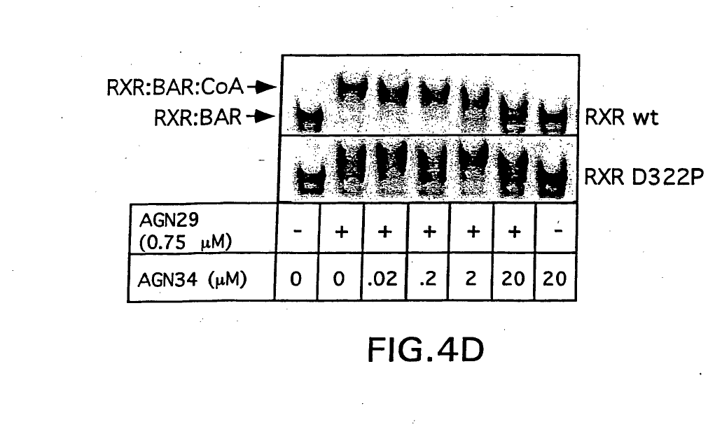

In the cotransfection assay AGN 34 did not affect the basal activity of luciferase gene expression, but repressed CDCA-activated BAR-mediated transcription nearly 10-fold (FIG. 4A). Dose-response analysis (increasing concentration of AGN 34; constant concentration of CDCA) in the same assay system indicated that AGN34 is a very strong repressor, displaying approximately 85% repression at 0.03 μM and showing maximal activity at 1 μM (FIG. 4B).

Various other nuclear receptor/RXR heterodimers were incubated with the cognate ligand of the non-RXR component of the heterodimer. These were: human AR (androgen receptor ), mouse PXR (10 μM pregnenolone-16-carbonitrile), human PXR (10 μM rifampicin), ERα (100 nM 17β-estradiol), human LXRα (30 μM hyodeoxycholic acid methyl ester), mouse PPARα (5 μM Wy 14,643), mouse PPARγ (1 μM rosiglitazone), mouse PPAγ (1 μM carbaprostacyclin), human VDR (100 nM 1,25-dihydroxyvitamin D₃) and human TRβ (100 nM triiodothyronine). No ligand was added to mouse CARβ which is constitutively active. Each compound was incubated in the presence or absence of 1 mM AGN 34, then assayed for luciferase reporter activity. As indicated in FIG. 4C, AGN34 was unable to repress the specific ligand-induced activity of these nuclear receptors, while AGN 34 remained able to repress the BAR/RXR heterodimer almost 10-fold.

We next tested the ability of AGN34 to displace the GRIP-1 co-activator from BAR. The results are shown in FIG. 4D. BAR:RXR:GRIP1 form a complex only in the presence of agonist; in this case 0.75 μM AGN 29 (lane 2). The addition of increasing concentrations of AGN 34 resulted in the progressive dissociation of that complex, suggesting that AGN 34 competes with agonists for binding to BAR. The experiments were repeated with RXRm, a mutant RXR in which asp322 within the RXR LBD has been replaced with a proline, rendering the RXRm with over 100-fold reduced Kd for ligand. Competition experiments using the BAR/RXRm heterodimer binds yielded the same results as obtained using the BAR/RXR heterodimer. (FIG. 4D).

To verify that AGN34 represses BAR/FXR in vivo, we looked at its effect on BAR/FXR target genes in cultured cells by Northern blot analysis. Differentiated Caco-2 cells and HepG2 cells were treated for 24 hours with 100 μM CDCA alone or in combination with 1 μM AGN34, then the RNA extracted and Northern blot performed as indicated above.

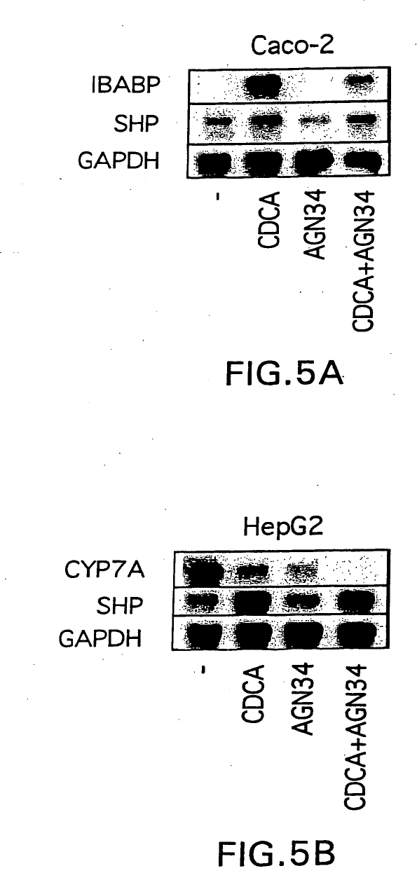

As expected, CDCA increased IBABP expression sharply in Caco-2 cells and the addition of AGN34 reduced the induced IBABP levels close to basal levels (FIG. 5A). To further confirm the role of AGN34, we looked at its effect on SHP expression both in Caco-2 and HepG2 cells. In both cell lines, AGN 34 did not affect basal SHP levels nor did it reduce CDCA-elevated expression. The effect of AGN 34 was also tested on CYP7A expression. In contrast to what would be expected from a BAR/FXR full antagonist, AGN 34 repressed rather than stimulated CYP7A expression, and this activity was repressed even further by the combination of AGN 34 and CDCA (FIG. 5B).

These results clearly indicate that AGN34 is a selective BAR/FXR modulator (BARM); that is, a BAR/FXR partial antagonist, that regulates different BAR/FXR target genes differentially. Antagonism of IBABP expression by AGN 34 is useful as a therapeutic method for the treatment of conditions such as colorectal cancer characterized by the presence of excessive levels of bile acid. Additionally, the fact that AGN 34 does not antagonize other BAR-regulated genes, such as Cyp 7A, means that such therapeutic use is quite specific and will therefore have a minimum of undesired side effects.

### EXAMPLE 4

To verify that AGN 34 modulates an activity that is characteristic of the BAR/FXR receptor, and that it does not function through the RXR homodimer, the following experiment was performed.

Luciferase reporter constructs (TK-luc) containing the Herpes virus thymidine kinase promoter (−105/+51) linked to the indicated number of copies of the following response elements: hsp27 EcRE×6 and MH100 ×4 (UAS_{G}×4) were used in a co-transfection assay conjunction with the following expression plasmids: GAL-L-hRXRα, hFXR, rFXRop, mFXR and hRXRop. As shown below, in most cases hRXRop was recombinantly coexpressed with one other of the indicated receptor constructs, so as to permit the formation of hetero- or homodimers containing hRXRop.

These constructs were tested in combination with the following test agents: CDCA, AGN 34, and various concentrations of these two agents. Methods are the same as for all transfections. Concentrations are noted in the second row of the table in μM. Except for the “none” column, all columns contained 50 μM CDCA +/− the indicated amount of AGN 34 in μM—e.g. last column is 50 μM CDCA+10 μM AGN34. Expression of the luciferase reporter gene product was detected and quantified as described above. The results are shown in the following table:

TABLE 1

| | Transfected | Transfected | | | AGN | CDCA + | CDCA + | CDCA + | CDCA + | CDCA + |
|---|---|---|---|---|---|---|---|---|---|---|
| Reporter | Receptor | Receptor | None | CDCA | 34 | AGN 34 | AGN 34 | AGN 34 | AGN 34 | AGN 34 |
| Ligand | | | | 50 | 10 | 0.01 | 0.1 | 1 | 3 | 10 |
| Amount | | | | | | | | | | |
| MH100 × 4 | GAL-L- | | 0.33 | 0.38 | 0.38 | 0.34 | 0.31 | 0.37 | 0.35 | 0.37 |
| | hRXRa | | | | | | | | | |
| EcRE × 6 | HFXR | HRXRop | 0.34 | 21.00 | 0.41 | 5.14 | 4.02 | 2.42 | 1.98 | 2.25 |
| EcRE × 6 | RFXRop | HRXRop | 0.32 | 19.29 | 0.27 | 2.92 | 2.05 | 1.28 | 0.98 | 1.28 |
| EcRE × 6 | MFXR | HRXRop | 0.22 | 17.71 | 0.26 | 4.37 | 2.45 | 1.38 | 1.29 | 1.36 |
| Fold | | | | | | | | | | |
| Activation | | | | | | | | | | |
| MH100 × 4 | GAL-L- | | 1.00 | 1.14 | 1.14 | 1.03 | 0.92 | 1.12 | 1.05 | 1.10 |
| | hRXRa | | | | | | | | | |
| EcRE × 6 | HFXR | HRXRop | 1.00 | 61.32 | 1.21 | 15.00 | 11.73 | 7.08 | 5.78 | 6.58 |
| EcRE × 6 | RFXRop | HRXRop | 1.00 | 59.59 | 0.84 | 9.03 | 6.32 | 3.94 | 3.04 | 3.94 |
| EcRE × 6 | MFXR | HRXRop | 1.00 | 79.50 | 1.18 | 19.61 | 10.99 | 6.19 | 5.77 | 6.11 |

These data indicate that when recombinant RXR is expressed alone (thus promoting the formation of RXR homodimers), the addition of CDCA, AGN 34 and the indicated concentrations of both ingredients does not result in the stimulation of cis-transactivation of the reporter gene. However, when human RXR is co-expressed with human, rat or mouse FXR (BAR) in transfected CV-1 cells , a resulting stimulation of gene expression is detected when the bile acid CDCA is added. AGN-34 alone does not cause a stimulation of transcription of the reporter gene. Increasing concentrations of AGN 34 added to a constant amount of CDCA in this system reduces the CDCA-mediated stimulation of reporter gene transcription in a dose-dependent manner. Thus, the data indicate that AGN 34 antagonizes the CDCA-mediated stimulation of BAR/FXR transcriptional activation, and that both the stimulation of transactivational activity by CDCA and the antagonism of this activity by AGN 34 is selective for the BAR/FXR receptor or the BAR:RXR heterodimer; neither agent shows activity in a system containing only RXR (and thus presumably only RXR homodimers.

The same reporter and expression constructs were then tested in the same manner in combination with the following test agents: AGN 34, LG 268, and various concentrations of AGN 34 and at a constant concentration of LG 268. LG 268 is an RXR agonist. The results are shown in the following table:

TABLE 2

| | | | | | AGN | LG268 + | LG268 + | LG268 + | LG268 + | LG268 + |
|---|---|---|---|---|---|---|---|---|---|---|
| | | | None | LG268 | 34 | AGN 34 | AGN 34 | AGN 34 | AGN 34 | AGN 34 |
| Reporter | | | | 0.1 | 10 | 0.01 | 0.1 | 1 | 3 | 10 |
| Activity | | | | | | | | | | |
| MH100 × 4 | GAL-L- | | 0.33 | 17.16 | 0.37 | 11.17 | 2.86 | 0.36 | 0.35 | 0.33 |
| | hRXRa | | | | | | | | | |
| EcRE × 6 | HFXR | HRXRop | 0.32 | 1.47 | 0.44 | 0.76 | 0.47 | 0.40 | 0.41 | 0.39 |
| EcRE × 6 | RFXRop | HRXRop | 0.28 | 11.94 | 0.29 | 5.24 | 0.44 | 0.25 | 0.26 | 0.25 |
| EcRE × 6 | MFXR | HRXRop | 0.25 | 12.95 | 0.35 | 6.73 | 0.39 | 0.27 | 0.26 | 0.29 |
| Fold | | | | | | | | | | |
| Activation | | | | | | | | | | |
| MH100 × 4 | GAL-L- | | 1.00 | 51.55 | 1.12 | 33.56 | 8.61 | 1.07 | 1.05 | 0.98 |
| | hRXRa | | | | | | | | | |
| EcRE × 6 | HFXR | HRXRop | 1.00 | 4.54 | 1.35 | 2.36 | 1.45 | 1.23 | 1.26 | 1.22 |
| EcRE × 6 | RFXRop | HRXRop | 1.00 | 42.42 | 1.02 | 18.59 | 1.55 | 0.88 | 0.92 | 0.90 |
| EcRE × 6 | MFXR | HRXRop | 1.00 | 52.10 | 1.39 | 27.08 | 1.56 | 1.07 | 1.04 | 1.18 |

As can be seen from the data, LG 268 stimulates RXR-mediated reporter gene transcription when used as the sole ligand in this experiment, and when RXR is the only receptor recombinantly expressed by the cell. LG 268 also stimulates transctivation of the reporter gene which RXR is co-expressed with rat or mouse BAR, but not with human recombinant BAR/FXR This result suggests that LG 268may have a measure of BAR/FXR (or BAR:RXR) agonist activity on the rat and mouse receptors as well as on RXR itself.

The addition of increasing concentrations of AGN 34 at a constant LG 268 concentration results in an attenuation of the LG 268-mediated transactivation activity in a manner consistent with antagonism of LG 268 activity. AGN 34 alone, does not stimulate receptor-mediated transactivation.

Subsequent gel shift experiments indicate that AGN 34 fully displaces associated co-activator (GRIP1) from BAR:RXR heterodimers in which LG 268 was added. Much lower doses of AGN 34 are necessary to cause full displacement than for the displacement of co-activator from RXR:BAR-CDCA complexes. Similar results are seen in a mammalian two hybrid assay detecting recruitment of CoA; AGN 34 inhibits recruitment of CoA, to the BAR:RXR heterodimer.

### EXAMPLE 5

An experiment conducted in a manner similar to that of Example 4 was carried out using either CDCA (50 μM) alone or in combination with AGN 34 (1 μM) or LG 754(1 μM) (an RXR homodimer antagonist but activator of PPAR-RXR) as test agents. The results are shown in the following table:

TABLE 3

| | | | | | CDCA + | CDCA + |
|---|---|---|---|---|---|---|
| Fold Activation | | | None | CDCA | AGN 34 | LG 754 |
| MH100 × 4 | GAL-L-hRXRa | | 1.00 | 0.96 | 0.38 | 0.79 |
| EcRE × 6 | HFXR | HRXRop | 1.00 | 31.41 | 5.51 | 16.67 |
| EcRE × 6 | RFXRop | HRXRop | 1.00 | 71.94 | 4.99 | 16.40 |
| EcRE × 6 | MFXR | HRXRop | 1.00 | 58.61 | 5.26 | 26.02 |

These results confirm, as above, that CDCA is able to stimulate BAR-mediated transactivation of the reporter gene. LG 754 antagonizes the CDCA-mediated activity, but to a much lesser degree than AGN 34.

The following example provides a detailed description of compounds having BAR/FXR modulating activity, as well as methods of making such compounds.

### EXAMPLE 6

### Synthesis of AGN 31

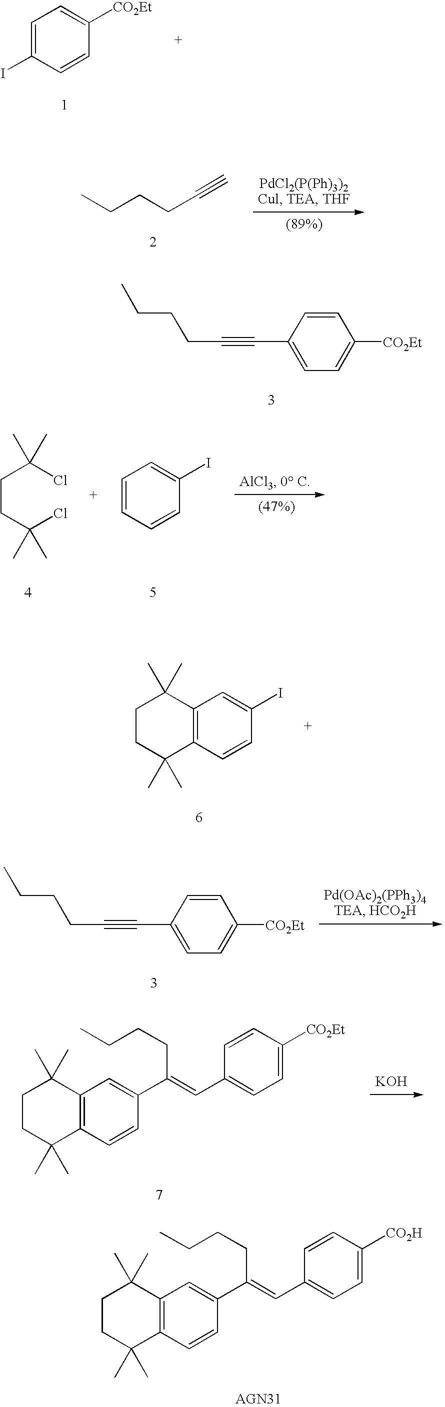

Ethyl 4-Hex-1-ynylbenzoate (3). A solution of 1-hexyne (1.72 mL, 15 mmol), ethyl 4-iodobenzoate (1.38 g, 5 mmol), triethylamine (1.05 mL, 7.5 mmol), and THF (20 mL) was degassed with argon for ten minutes. The solution was treated with bis(triphenylphosphine)palladium (II) chloride (17.5 mg, 0.25 mmol) and copper iodide (11.4 mg, 0.06 mg) and it was stirred at room temperature for 24 h. The solution was concentrated under reduced pressure, and the residue was purified by silica gel chromatography hexane) to give the title compound. HNMR (CDCl₃, 300 MHz): δ0.95 (t, 3H, J=7.3 Hz), 1.39 (t, 3H, J=7.1 Hz), 1.49 (m, 2H), 1.59 (m, 2H), 2.43 (t, 2H, J=7.1 Hz), 4.36 (q, 2H, J=7.3 Hz), 7.44 (d, 2H, J=8.3 Hz), 7.95 (d, 2H, J=8.3 Hz).

6-Iodo-1,1,4,4-tetramethyl-1,2,3,4-tetrahydronaphthalene (6). Aluminum trichloride was added very slowly to an ice-cold solution of iodobenzene (6.1 mL, 54.6 mmol) and 2,5-dichloro-2,5-dimethylhexane (5 g, 27.3 mmol). After 20 minutes, the solution was diluted with hexane and poured over ice water. The layers were separated and the aqueous layer extracted two times with hexane. The combined organic layers were washed with water and brine, dried over MgSO₄, and the filtered solvents were removed under reduced pressure. The excess iodobenzene was removed under high vacuum to give the title compound as a colorless solid. HNMR (CDCl₃, 300 MHz): δ0.1.25 (s, 6H), 1.26 (s, 6H), 1.65 (s, 2H), 1.66 (s, 2H), 7.03 (d, 1H, J=8.3 Hz), 7.42 (dd, H, J=2.0, 8.3 Hz), 7.59 (d, 1H, J=2.0 Hz).

4-[2-(5,5,8,8-Tetramethyl-5,6,7,8-tetrahydronaphthalen-2-yl)hex-1-enyl]benzoic Acid (AGN 31). Ethyl 4-hex-1-ynylbenzoate (192 mg, 0.83 mmol) and 6-iodo-1,1,4,4-tetramethyl-1,2,3,4-tetrahydronaphthalene (628 mg, 2 mmol) were mixed with triethylamine (0.4 mL). Bis(triphenylphosphine)palladium (II) diacetate (16 mg, 0.021 mmol) and acetonitrile (0.3 mL) were added, and the solution was purged with argon for a few minutes. Acetic acid (0.083 mL, 2.20 mmol) was added and the solution was stirred overnight at 80° C. The reaction was diluted with water and ether, the layers were separated, and the aqueous layer extracted twice with ether. The combined ether layers were washed with brine, and dried (MgSO4), and filtered, and the solvent was removed under reduced pressure. The residue was purified by silica gel chromatography using 9:1 hexane:ethyl acetate as the eluent to give an inseparable mixture of isomeric esters. The esters were hydrolyzed with 2N aqueous KOH (1 mL) in ethanol (4 mL), acidified with 1N HCl, and the products were extracted with ethyl acetate. The organic extracts were washed with brine and dried (MgSO4). The filtered solvent was removed in vacuo and the resulting solid recrystalized from a solution of hexane and ethyl acetate to give the title compound. HNMR (CDCl₃, 300 MHz): δ0.882 (t, 3H, J=7.1 Hz), 1.22-1.55 (m, 4H), 1.31(s, 6H), 1.32 (s, 6H), 1.71 (s, 4H), 2.69 (t, 2H, J=8.3 Hz), 6.72 (s, 1H), 7.24 (d, 1H, J=8.2 Hz), 7.30 (d, 1H, J=8.3 Hz), 7.40 (s, 1H), 7.42 (d, 1H, J=8.4 Hz), 8.11 (d, 1H, J=8.4 Hz).

### EXAMPLE 7

### Synthesis of AGN 34

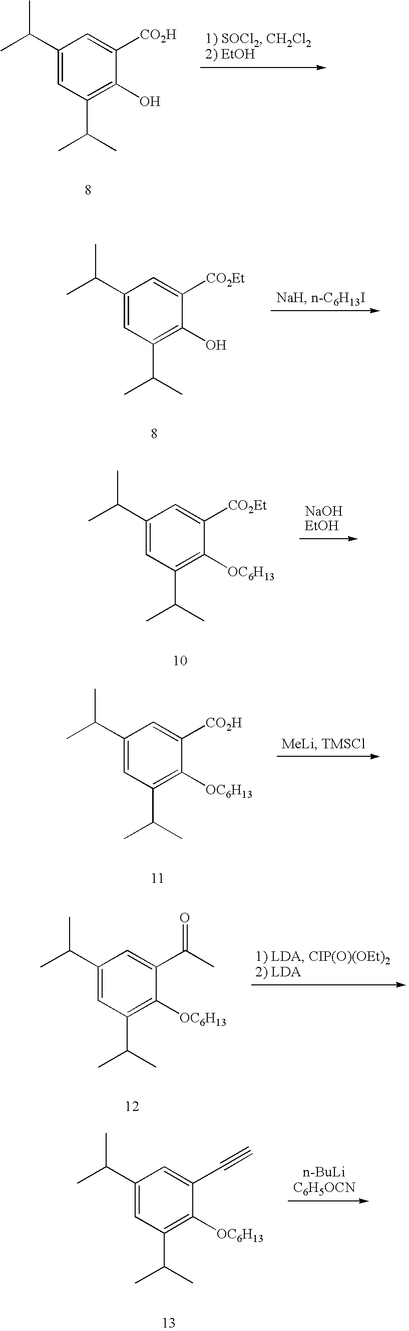

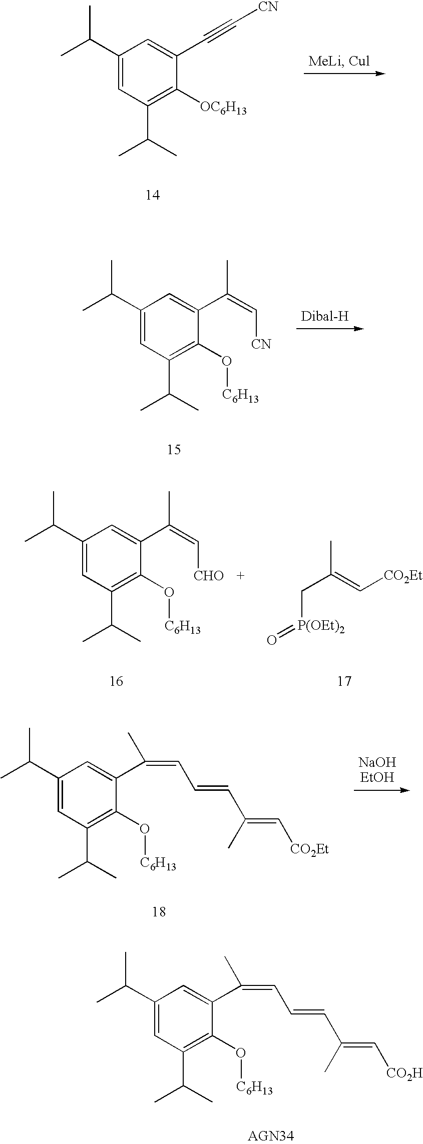

Ethyl 2-Hydroxy-3,5-diisopropylbenzoate (9). Thionyl chloride (48 mL, 675 mmol) was added to a solution of 2-hydroxy-3,5-diisopropylbenzoic acid (15 g, 67.5 mmol) and dichloromethane (60 mL), and the resulting solution was heated to reflux for 18 hours. The solution was cooled to room temperature and the solvents were removed under vacuum. Ethanol was added and the solution was stirred at room temperature for 4 hours. The solvent was evaporated, and the residue was purified by silica gel chromatography (hexane:ethyl acetate::4:1). HNMR (CDCl₃, 300 MHz): δ1.24 (d, 6H, J=3.6 Hz), 1.27 (d, 6H, J=3.6 Hz), 1.43 (t, 3H, J=7.0 Hz), 2.87 (m, 1H, J=3.6 Hz), 3.37 (m, 1H, J=3.6 Hz), 4.42 (q, 2H, J=7.0 Hz), 7.27 (s, 1H), 7.55 (s, 1H), 11.1 (s, 1H).

Ethyl 2-Hexyloxy-3,5-diisopropylbenzoate (10). A solution of ethyl 2-hydroxy-3,5-diisopropylbenzoate (19.1 g, 76.4 mmol) and 10 mL DMF was added slowly to a suspension of sodium hydride (4 g, 99.3 mmol) and DMF (90 mL) at 0° C. After 10 minutes, 1-iodohexane (17.0 mL, 114.6 mmol) was added, and the solution was stirred at room temperature for 18 hours. The reaction was quenched by the addition of water and the products extracted with ethyl acetate. The organic layers were combined, and washed with brine, and dried over MgSO4. The filtered solvents were removed by rotary evaporation to give the title compound as a yellow oil that was used in the next step without further purification. HNMR (CDCl₃, 300 MHz): δ0.91 (t, 3H, J=7.2 Hz), 1.15-1.53 (m, 6H), 1.23 (d, 12H, J=6.9Hz), 1.41 (t, 3H, J=7.2 Hz), 1.81 (m, 3H, J=7.2 Hz), 2.89 (m, 1H, J=7.2Hz),3.39(m, 1H, J=7.2 Hz), 3.85 (t, 2H, J=6.9 Hz), 4.39 (q, 2H, J=6.9 Hz), 7.25 (d, 1H, J=2.4 Hz), 7.45 (d, 1H, J=2.4 Hz).

2-Hexyloxy-3,5-diisopropylbenzoic Acid (11). A solution of ethyl 2-hydroxy-3,5-diisopropylbenzoate (25.5 g, 76.4 mmol) and ethanol (200 mL) was treated with 2N aqueous NaOH (50 mL, 100 mmol). The solution was stirred at room temperature for 3 days, and acidified with 2N HCl, and the products were extracted with ethyl acetate. The combined organic extracts were washed with brine, and dried over MgSO4. The filtered solvent was concentrated under reduced pressure, and the residue was purified by silica gel chromatography (ethyl acetate:hexane::3:2) to give the title compound as a yellow solid. HNMR (CDCl₃, 300 MHz): δ0.91 (t, 3H, J=7.2 Hz), 1.18-1.42 (m, 4H), 1.24 (d, 6H, J=6.9 Hz), 1.26 (d, 6H, J=6.9 Hz), 1.48 (m, 2H), 1.88 (m, 3H, J=7.2 Hz), 2.92 (m, 1H, J=6.9 Hz), 3.28 (m, 1H, J=6.9 Hz), 3.92 (t, 2H, J=6.9 Hz), 7.34 (d, 1H, J=2.4 Hz), 7.82 (d, 1H, J=2.4 Hz).

2-Hexyloxy-3,5-diisopropylbenzophenone (12). A solution of CH₃Li in ether (1.4 M, 48 mL, 67.2 mmol) was added to a solution of 2-hexyloxy-3,5-diisopropylbenzoic acid (5.2 g, 16.9 mmol) and THF (100 mL) at 0° C. under argon. The solution was stirred at 0° C. for 3.5 hours and treated with trimethylsilyl chloride (50 mL). The resulting cloudy solution was stirred for 20 minutes at room temperature and then the reaction was quenched by the addition of 2N HCl. After stirring the solution for an hour at room temperature, the product was extracted with ethyl acetate three times. The extracts were combined and washed with brine, and dried (MgSO4), and filtered, and the solvents were concentrated in vacuo to give the title compound as a brown oil, which was not further purified. HNMR (CDCl₃, 300 MHz): δ0.91 (t, 3H, J=6.9 Hz), 1.16-1.42 (m, 4H), 1.24 (d, 12H, J=7.2 Hz), 1.45 (m, 2H), 1.79 (m, 3H, J=7.2 Hz), 2.63 (s, 3H), 2.89 (m, 1H, J=7.2 Hz), 3.35 (m, 1H, J=6.9Hz), 3.72 (t, 2H, J=7.2 Hz), 7.21 (d, 1H, J=2.1 Hz), 7.24 (d, 1H, J=2.1 Hz).

1-Ethynyl-2-hexyloxy-3,5-diisopropylbenzene (13). To a solution of diisopropylamine (3.4 mL, 24.2 mmol) in THF (20 mL) at 0° C. was added a solution of n-BuLi in hexane (1.6 M, 17 mL, 27.4 mmol). The solution was stirred at 0° C. for 30 minutes and then cooled to −78° C. A solution of 2-Hexyloxy-3,5-diisoprpoylbenzophenone (4.9 g, 16.1 mmol) and THF (20 mL) was added, and the solution was stirred for 1 hour at −78° C. Diethyl chlorophosphate (3.1 mL, 20.9 mmol) was added to the solution and the reaction was allowed to slowly warm up to room temperature over three hours. A second solution of lithium diisopropylamine (LDA) was prepared as described above by adding n-BuLi (33 mL, 82.2 mmol) to a solution of diisopropylamine (10.2 mL, 72.6 mmol) and THE (20 mL) at 0° C. and then cooling this solution to −78° C. The first solution was added to the LDA at −78° C., and the resulting solution was allowed to warm to room temperature over three hours. Adding water and ethyl acetate quenched the reaction. The layers were separated and the aqueous layer was extracted two times with ethyl acetate. The combined extracts were washed with brine, dried over MgSO4, filtered and concentrated under reduced pressure. The residue was purified by flash chromatography using hexane and ethyl acetate in a 4:1 ratio to give the title compound as a yellow oil. HNMR (CDCl₃, 300 MHz): δ0.92 (t, 3H, J=6.9 Hz), 1.13-1.45 (m, 4H), 1.22 (d, 6H, J =7.2 Hz), 1.23 (d, 6H, J=7.2 Hz), 1.50 (m, 2H), 1.82 (m, 3H, J=7.2 Hz), 2.84 (m, 1H, J=6.9 Hz), 3.22 (s, 1H), 3.33 (m, 1H, J=6.9 Hz), 4.03 (t, 2H, J=6.6 Hz), 7.08 (d, 1H, J=2.1 Hz), 7.17 (d, 1H, J=2.1 Hz).

Cyanatobenzene. A solution of cyanogen bromide (24.3 g, 230 mmol) and water (75 mL) was added to a solution of phenol (20.7 g, 220 mmol) and carbon tetrachloride (75 mL) at 0° C. The solution was treated with triethylamine (31 mL, 220 mmoL) over 30 minutes, and then it was allowed to warm to room temperature and stirred for 18 hours. The mixture was diluted with ethyl acetate, and washed with brine, and dried (MgSO4), and filtered, and the solvent was removed by rotary evaporation. The residue was purified by flash chromatography on silica gel (4:1/hexane:ethyl acetate) to give the title compound as a clear oil. HNMR (CDCl₃, 300 MHz): δ7.34 (br s, 3H), 7.45 (br s, 2H).

(2-Hexyloxy-3,5-diisopropylphenyl)propynenitrile (14). A solution of n-BuLi in hexanes (1.6 M, 3.2 mL, 8.06 mmol) was added slowly to a solution of 1-ethynyl-2-hexyloxy-3,5-diisopropylbenzene (2.1 g, 7.33 mmol) and THF (20 mL) at −78° C. The solution was stirred at −78° C. for ten minutes and treated with a solution of cyanatobenzene (1.0 g, 8.06 mmol) and THF (3 mL). The solution was warmed to room temperature over two hours, and then 2N NaOH was added. The products were extracted with ethyl acetate (3×), the organic layers were combined and washed with brine and dried over MgSO₄. The filtered solvents were concentrated under reduced pressure and the residue was purified by flash chromatography (SiO₂, 4:1/hexane:ethyl acetate) to give the title compound as a yellow oil. HNMR (CDCl₃, 300 MHz): δ0.94 (t, 3H, J=6.9 Hz), 1.13-1.40 (m, 4H), 1.22 (d, 6H, J=6.9 Hz), 1.23 (d, 6H, J=6.9 Hz), 1.53 (m, 2H), 1.83 (m, 3H), 2.85 (m, 1H, J=6.9 Hz), 3.30 (m, 1H, J=6.9 Hz), 3.99 (t, 2H, J=6.6 Hz), 7.24 (s, 1H), 7.24 (s, 1H).

(Z)-3-(2-Hexyloxy-3,5-diisopropylphenyl)but-2-enenitrile (15). A solution of MeLi and ether (1.4 M, 27.5 mL, 38.5 mmol) was added to a stirring solution of copper iodide (3.70 g, 19.2 mL) in THF (50 mL) at 0° C. The solution was cooled to −78° C. and a solution of (2-hexyloxy-3,5-diisopropylohenyl)propynenitrile (2.99 g, 9.62 mmol) in THF (5 mL) was added slowly. The solution was stirred at −78° C. for 1 hour and then quenched by the addition of 15 mL of methanol. The products were extracted with ethyl acetate and saturated aqueous NH₄Cl. The combined organic layers were washed with brine and dried over MgSO₄. The filtered solvent was concentrated under reduced pressure and the concentrate was purified by silica gel chromatography using a 95:5 mixture of hexane:ethyl acetate to produce the title compound as a yellow oil. HNMR (CDCl₃, 300 MHz): δ0.92 (t, 3H, J=6.6 Hz), 1.13-1.40 (m, 4H), 1.24 (d, 6H, J=6.6 Hz), 1.25 (d, 6H, J=6.9 Hz), 1.44 (m, 2H), 1.72 (m, 3H, J=6.9 Hz), 2.30 (d, 3H, J=1.5 Hz), 2.89 (m, 1H, J=6.9 Hz), 3.32 (m, 1H, J=6.9 Hz), 3.70 (t, 2H, J=6.6 Hz), 5.42 (d, 1H, J=1.5 Hz), 6.92 (d, 1H, J=2.1 Hz), 7.12 (d, 1H, J=2.1 Hz).

(Z)-3-(2-Hexyloxy-3,5-diisopropylphenyl)but-2-enal (16). A solution of DIBAL-H in dichloromethane (1.0 M, 4.50 mL, 4.50 mmol) was added to a solution of (Z)-3-(2-hexyloxy-3,5-diisopropylphenyl)but-2-enenitrile (1.02 g, 3.12 mmol) and hexane (30 mL) at −78° C. The solution was stirred at −78° C. for six hours, and a solution of 20% sodium potassium tartrate was added at −78° C. The solution was warmed to room temperature and the products extracted with ethyl acetate (3×). The combined organic layers were washed with brine, and dried (MgSO₄), and filtered. The solvent was removed by rotary evaporation and the residue purified by flash chromatography on silica gel using a 95:5 mixture of hexane and ethyl acetate to give the title compound as yellow oil. HNMR (CDCl₃, 300 MHz): δ0.91 (t, 3H, J=6.3 Hz), 1.13-1.40 (m, 4H), 1.23 (d, 6H, J=6.6 Hz), 1.25 (d, 6H, J=6.6 Hz), 1.44 (m, 2H), 1.72 (m, 3H, J=6.9 Hz), 2.30 (s, 3H), 2.89 (m, 1H, J=6.6 Hz), 3.32 (m, 1H, J =6.6 Hz), 3.64 (t, 2H, J=6.6 Hz), 6.21 (d, 1H, J=8.4 Hz), 6.81 (d, 1H, J=2.4 Hz), 7.12 (d, 1H, J=2.4 Hz), 9.44 (d, 1H, J=8.4 Hz).

Ethyl (2E, 4E, 6Z)-7-(2-Hexyloxy-3,5-diisopropylphenyl)-3-methylocta-2,4,6-trienoate (18). A solution of n-BuLi in hexanes (2.5 M, 6.4 mL, 15.9 mmol) was added to a solution of DMPU (4.0 mL) and Ethyl (E)-4-(diethoxyphosphoryl)-3-methylbut-2-enoate¹ (4.2 g, 15.9 mmol) and THF (20 mL) at −78° C. After 15 minutes, a solution of (Z)-3-(2-hexyloxy-3,5-diisopropylphenyl)but-2-enal (1.05 g, 3.18 mmol) and THF (5 mL) was added dropwise over 10 to 15 minutes, and the solution was warmed to 0° C. and stirred for one hour. The reaction was quenched at 0° C. by the addition of aqueous NH₄Cl. The products were extracted with ethyl acetate and the combined organic layers were washed with brine and dried over MgSO₄. The filtered solvent was concentrated under reduced pressure and the concentrate was purified by silica gel chromatography using a 90:10 mixture of hexane:ethyl acetate to produce the title compound as a yellow oil. HNMR (CDCl₃, 300 MHz): δ0.89 (t, 3H, J=6.6 Hz), 1.13-1.45 (m, 4H), 1.23 (d, 6H, J=6.9 Hz), 1.25 (d, 6H, J=6.9 Hz), 1.28 (t, 3H, J=7.1 Hz), 1.39 (m, 2H), 1.65 (m, 3H, J=6.6 Hz), 2.15 (s, 314), 2.21 (s, 3H), 2.86 (m, 1H, J=6.9 Hz), 3.34 (m, 1H, J=6.9 Hz), 3.63 (t, 2H, J=6.3 Hz), 4.15 (q, 2H, J=7.2 Hz), 5.74 (s, 1H), 6.21 (d, 1H, J=15.4 Hz), 6.22 (d, 1H, J=10.5 Hz), 6.50 (dd, 1H, J=10.5, 15.4 Hz), 6.75 (d, 1H, J=2.3 Hz), 7.04 (d, 1H, J=2.3 Hz).

(2E, 4E, 6Z)-7-(2-Hexyloxy-3,5-diisopropylphenyl)-3-methylocta-2,4,6-trienoic Acid (AGN 34). A solution of ethyl (2E, 4E, 6Z)-7-(2-hexyloxy-3,5-diisopropylphenyl)-3-methylocta-2,4,6-trienoate (1.2 g, 2.73 mmol) and ethanol (40 mL) was treated with 2N aqueous NaOH (30 mL, 60 mmol). The solution was stirred at 60° C. for 18 hours, acidified with 2N HCl, and the products were extracted with ethyl acetate. The combined organic extracts were washed with brine, and dried over MgSO₄. The filtered solvent was concentrated under reduced pressure, and the residue was purified by recrystalization from ethanol to give the title compound as a yellow solid. HNMR (CDCl₃, 300 MHz): δ0.89 (t, 3H, J=6.3 Hz), 1.13-1.45 (m, 4H), 1.22 (d, 6H, J=6.9 Hz), 1.24 (d, 6H, J=6.9 Hz), 1.29 (m, 2H), 1.65 (m, 3H, J=6.3 Hz), 2.15 (s, 3H), 2.21 (s, 3H), 2.86 (m, 1H, J=6.9 Hz), 3.33 (m, 1H, J=6.9 Hz), 3.62 (t, 2H, J=6.3 Hz), 5.75 (s, 1H), 6.23 (d, 1H, J=15.3 Hz), 6.25 (d, 1H, J=10.8 Hz), 6.62 (dd, 1H, J=10.8, 15.3 Hz), 6.74 (d, 1H, J=2.1 Hz), 7.03 (d, 1H, J=2.1 Hz).

### EXAMPLE 8

### Synthesis of AGN 29

4-Bromobenzyl tert-butyldiphenylsilyl ether

Tert-butyldiphenylsilyl chloride (10.4 mL, 40.1 mmol) was added to a solution of 4-bromobenzyl alcohol ( 5.0 g, 26.7 mmol) and 50 mL of dichloromethane. The solution was treated with triethylamine (3.72 mL, 26.7 mmol) and (dimethylamino)pyridine (163 mg, 1.34 mmol) and stirred overnight at room temperature. The solution was diluted with 300 mL of dichloromethane and washed with 50 mL of 10% aqueous HCl. The layers were separated and the aqueous layer was extracted with 50 mL of dichloromethane. The combined organic extracts were washed with brine, and dried (MgSO₄), and filtered, and the solvents were removed in vacuo. The residue was filtered through a plug (6@×2@) of silica gel using a solution of 97% hexane/ethyl acetate. After removal of the solvent the residue was heated under vacuum (3 torr) to 170° C. for 1 hour to remove a low-boiling impurity. The remaining material is the title compound. PNMR (300 MHz, CDCl₃) δ1.09 (s, 9H), 4.70 (s, 2H), 7.20 (d, 2H, J=7.9 Hz), 7.35-7.45 (m, 8H), 7.65 (overlapping ds, 4H).

4-[(trimethylsilyl)ethynyl]benzyl tert-butyldiphenylsilyl ether

A 25 mL round bottom flask was flame-dried under high vacuum. The vacuum was broken by the addition of dry argon, and the flask was allowed to cool to room temperature. The flask was charged with 2.0 g (4.70 mmol) of 4-bromobenzyl tert-butyldiphenylsilyl ether (Compound 1), 2.0 mL (14.1 mmol) of (trimethylsilyl)acetylene, and 16.5 mL of triethylamine. The solution was purged with argon for 15 minutes and bis(triphenylphosphine)palladium (II) chloride (83 mg, 0.12 mmol) and copper (I) iodide (22 mg, 0.12 mmol) were added and the solution stirred at ambient temperature for 3 days. The solution was poured into a separatory funnel containing water and ether. The layers were separated and the aqueous layer was extracted 3 times with ether. The combined ether layers were washed once with brine, and dried over magnesium sulfate, and the solvents were removed under reduced pressure. The residue was purified by distillation (bp=180°0 B 185° C., 1 torr) to give the title compound. PNMR (300 MHz, CDCl₃) δ0.23 (s, 9H), 1.09 (s, 9H), 4.73 (s, 2H), 7.23 (d, 2H, J=7.9 Hz), 7.31-7.45 (m, 8H), 7.65 (overlapping ds, 4H).

(Z)-4-[2-(3,5,5,8,8-Pentamethyl-5,6,7,8-tetrahydronaphthalen-2-yl)-2-(trimethylsilyl)vinyl]benzyl alcohol A 3-neck 25 mL round bottom flask was fitted with a reflux condenser, and flame-dried under high vacuum. The vacuum was broken by the addition of dry argon (3×), and the flask was allowed to cool to room temperature. The flask was charged with 0.5 mL (1.0 mmol) of borane-methyl sulfide and THF (0.3 mL) and cooled to 0° C. The solution was treated with 0.20 mL (2 mmol) of cyclohexene and stirred at 0° C. for 1 hour. Neat 4-[(trimethylsilyl)ethynyl]benzyl tert-butyldiphenylsilyl ether (443 mg, 1 mmol) was added and, after 15 minutes the solution was warmed to room temperature and stirred for 2.25 hours. In a second flask was prepared a solution of tetrakis(triphenylphosphine)palladium (0) (58 mg, 0.05 mmol) and 2-bromo-3,5,5,8,8-pentamethyl-5,6,7,8-tetrahydronaphthalene (1.26 g, 4.5 mmol) in 5 mL of THF, which was purged with argon for 10 minutes. The solvents in the first flask were removed under high vacuum, and the residue dissolved in 1 mL of THEF and 1 mL of 2 M aqueous NaOH, and the resulting solution was purged with argon for 10 minutes. A 1 mL aliquot of the solution from the second flask was added to the first flask, and the reaction was protected from light and refluxed for 5 hours. The reaction was cooled to room temperature and treated with 2 M NaOH (1 mL) and 30% hydrogen peroxide (0.4 mL). The solution was poured into a separatory funnel containing water and pentane. The layers were separated and the aqueous layer was extracted 3 times with pentane. The combined organic layers were washed once with brine, and dried over magnesium sulfate, and the solvents were removed under reduced pressure. The residue was partially purified by silica gel chromatography (99:1, hexane:ethyl acetate). The later fractions were combined and concentrated under reduced pressure. The residue (203 mg) was dissolved in 3.2 mL of THF and treated with 313 mg of tetrabutylammonium fluoride (Tbaf) adsorbed onto silica gel (1.6 mmol fluoride per gram). The suspension was stirred for 5 hours at room temperature and then the silica gel was washed with ether, and the separated ether extracts were dried over magnesium sulfate. The filtered solvents were removed under reduced pressure and the residue purified by silica gel chromatography (4:1, hexane:ethyl acetate) to give the title compound. PNMR (300 MHz, CDCl₃) −0.10 (s, 9H), 1.29 (s, 12H), 1.68 (s, 4H), 2.24 (s, 3H), 4.72 (s, 2H), 6.87 (s, 1H), 7.07 (s, 1H), 7.17 (s, 1H), 7.35 (s, 4H).

Ethyl (Z)-4-[2-(3,5,5,8,8-pentamethyl-5,6,7,8-tetrahydronaphthalen-2-yl)-2-(trimethylsilyl)vinyl]benzoate Manganese dioxide (265 mg, 2.96 mmol) was added to a solution of (Z)-4-[2-(3,5,5,8,8-pentamethyl-5,6,7,8-tetrahydronaphthalen-2-yl)-2-(trimethylsilyl)vinyl]benzyl alcohol (60 mg, 0.15 mmol) and 3.65 mL of hexane. The solution was stirred at room temperature for 16 hours, the manganese dioxide filtered off, and the hexane removed in vacuo. The residue was dissolved in 2 mL of ethanol and treated with sodium cyanide (37.5 mg, 0.77 mmol) and acetic acid (13.7 mg, 0.23 mmol). After 15 minutes, the solution was treated with 265 mg (3.0 mnmol) of manganese dioxide. The suspension was stirred at room temperature for 6 hours and the manganese dioxide removed by filtration. The solution was poured into a separatory funnel containing water and ether. The layers were separated and the aqueous layer was extracted 3 times with ether. The combined organic layers were washed once with brine, and dried over magnesium sulfate, and the solvents were removed under reduced pressure. The residue was purified by silica gel chromatograhy (97:3, hexane:ethyl acetate) to give the title compound. PNMR (300 MHz, CDCl₃) −0.11 (s, 9H), 1.28 (s, 12H), 1.41 (t, 3H, J=7.1 Hz), 1.68 (s, 4H), 2.23 (s, 3H), 4.39 (q, 2H, J=7.1 Hz), 6.86 (s, 1H), 7.08 (s, 1H), 7.17 (s, 1H), 7.41 (d, 2H, J=8.5 Hz), 8.03 (d, 2H, J=8.5 Hz).

(Z)-4-[2-(3,5,5,8,8-Pentamethyl-5,6,7,8-tetrahydronaphthalen-2-yl)-2-(trimethylsilyl)vinyl]benzoic Acid (AGN 29) To a solution of ethyl (Z)-4-[2-(3,5,5,8,8-pentamethyl-5,6,7,8-tetrahydronaphthalen-2-yl)-2-(trimethylsilyl)vinyl]benzoate (0.034 g, 0.076 mmol) and 2 mL of ethyl alcohol was added aqueous 1 N KOH (0.5 mL). The resulting solution was heated in an 50° C. bath until the hydrolysis reaction was completed, as judged by thin layer chromatography. The solution was cooled to room temperature, diluted with water and washed once with 1:1 ether:hexane solution, and the layers were separated. The aqueous layer was acidified with 1 N aqueous HCl and the product extracted 3 times with ethyl acetate. The combined organic extracts were washed with brine, and dried over MgSO₄, and filtered, and the solvents were removed in vacuo to give AGN 29 as a white solid. PNMR (300 MHz, CDCl₃) δ−0.09 (s, 9H), 1.28 (s, 12H), 1.68 (s, 4H), 2.24 (s, 3H), 6.86 (s, 1H), 7.08 (s, 1H), 7.18 (s, 1H), 7.46 (d, 2H, J=8.1 Hz), 8.11 (d,2H, J=8.1 Hz).

Further disclosure can be found in U.S. Pat. No. 5,675,033 and in International Patent Application No. WO00/77011, both of which are hereby incorporated by reference herein. The latter publication also discloses the synthesis of AGN 29.

The examples set forth herein are meant to be illustrative only, and are not intended to limit the scope of the invention, which should be defined solely with reference to the claims that conclude this specification.

1. A method of treating a pathological condition in a mammal characterized by hypocholesterolemia comprising the step of providing to said mammal a pharmaceutically acceptable composition comprising a synthetic BAR/FXR ligand selected from the group consisting of AGN 29 and AGN 31.

2. The method of claim 1 wherein said composition comprises AGN 29.

3. The method of claim 1 wherein said composition comprises AGN 31.

4. A method of treating a pathological condition in a mammal characterized by pathological expression of IBABP, comprising the step of providing to said mammal a pharmaceutically acceptable composition comprising AGN 34, thereby treating said condition.

5. The method of claim 4 wherein said pathological condition comprises hypocholesterolemia.

6. The method of claim 4 wherein said pathological condition comprises colon cancer.

7. The method of claim 4 wherein said pathological condition is. characterized by high levels of bile acids.

8. A method of treating colon cancer in a mammal without increasing the expression of Cyp7A comprising the step of providing to said mammal a pharmaceutically acceptable composition comprising AGN 34.

9. The method of claim 8 in which AGN 34 stimulates the induction of apoptosis of colon cancer cells.

10. A method of inhibiting the bile-acid mediated growth of cancer cells in a mammalian cell comprising administering a composition comprising a pharmaceutically effective amount of AGN 34.


SEQUENCE LISTING (14 sequences)

SEQ ID NO: 1 - protein, 469 aa, Rattus norvegicus
MNLIGPSHLQATDEFALSENLFGVLTEHAAGPLGQNLDLESYSPYNNVQFPQVQPQISSSSYYSNLGFYPQQPEDWYSPGLYELRRMPTESVYQGETEVSEMPVTKKPRMAASSAGRIKGDELCVVCGDRASGYHYNALTCEGCKGFFRRSITKNAVYKCKNGGNCVMDMYMRRKCQDCRLRKCREMGMLAECLLTEIQCKSKRLRKNVKQHADQTVNEDSEGRDLRQVTSTTKLCREKTELTVDQQTLLDYIMDSYSKQRMPQEITNKILKEEFSAEENFLILTEMATSHVQILVEFTKRLPGFQTLDHEDQIALLKGSAVEAMFLRSAEIFNKKLPAGHADLLEERIRKSGISDEYITPMFSFYKSVGELKMTQEEYALLTAIVILSPDRQYIKDREAVEKLQEPLLDVLQKLCKIYQPENPQHFACLLGRLTELRTFNHHHAEMLMSWRVNDHKFTPLLCEIWDVQ

SEQ ID NO: 2 - protein, 484 aa, Mus musculus
MVMQFQGLENPIQISLHHSHRLSGFVPDGMSVKPAKGMLTEHAAGPLGQNLDLESYSPYNNVPFPQVQPQISSSSYYSNLGFYPQQPEDWYSPGIYELRRMPAETGYQGETEVSEMPVTKKPRMAAASAGRIKGDELCVVCGDRASGYHYNALTCEGCKGFFRRSITKNAVYKCKNGGNCVMDMYMRRKCQECRLRKCREMGMLAECLLTEIQCKSKRLRKNVKQHADQTVNEDDSEGRDLRQVTSTTKFCREKTELTADQQTLLDYIMDSYNKQRMPQEITNKILKEEFSAEENFLILTEMATSHVQILVEFTKKLPGFQTLDHEDQIALLKGSAVEAMFLRSAEIFNKKLPAGHADLLEERIRKSGISDEYITPMFSFYKSVGELKMTQEEYALLTAIVILSPDRQYIKDREAVEKLQEPLLDVLQKLCKMYQPENPQHFACLLGRLTELRTFNHHHAEMLMSWRVNDHKFTPLLCEIWDVQ

SEQ ID NO: 3 - protein, 476 aa, Homo Sapiens
PRTHMGSKMNLIEHSHLPTTDEFSFSENLFGVLTEQVAGPLGQNLEVEPYSQYSNVQFPQVQPQISSSSYYSNLGFYPQQPEEWYSPGIYELRRMPAETLYQGETEVAEMPVTKKPRMGASAGRIKGDELCVVCGDRASGYHYNALTCEGCKGFFRRSITKNAVYKCKNGGNCVMDMYMRRKCQECRLRKCKEMGMLAECLLTEIQCKSKRLRKNVKQHADQTVNEDSEGRDLRQVTSTTKSCREKTELTPDQQTLLHFIMDSYNKQRMPQEITNKILKEEFSAEENFLILTEMATNHVQVLVEFTKKLPGFQTLDHEDQIALLKGSAVEAMFLRSAEIFNKKLPSGHSDLLEERIRNSGISDEYITPMFSFYKSIGELKMTQEEYALLTAIVILSPDRQYIKDREAVEKLQEPLLDVLQKLCKIHQPENPQHFACLLGRLTELRTFNHHHAEMLMSWRVNDHKFTPLLCEIWDVQ

SEQ ID NO: 4 - protein, 462 aa, Homo Sapiens
MDTKHFLPLDFSTQVNSSLTSPTGRGSMAAPSLHPSLGPGIGSPGQLHSPISTLSSPINGMGPPFSVISSPMGPHSMSVPTTPTLGFSTGSPQLSSPMNPVSSSEDIKPPLGLNGVLKVPAHPSGNMASFTKHICAICGDRSSGKHYGVYSCEGCKGFFKRTVRKDLTYTCRDNKDCLIDKRQRNRCQYCRYQKCLAMGMKREAVQEERQRGKDRNENEVESTSSANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 5 - protein, 147 aa, S. Cerevisiae GAL4 DNA Binding Domain
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 6 - DNA, 39 bp, Artificial Sequence
  Linker
gtatcgccggaattcggtaccgtcgaggccgtgcaggag

SEQ ID NO: 7 - DNA, 56 bp, Artificial Sequence
  Linker
gtatcgccggaattcgggctaaggaagtgcagagagatgggaatgttggctgaatg

SEQ ID NO: 8 - protein, 9 aa, SV 40 Tag
APKKKRKVG

SEQ ID NO: 9 - DNA, 360 bp, Artificial Sequence
  TK-luc promoter
ggttttcccagtcacgacgttgtaaaacgacggccagtgccaagcttgcatgcctgcaggtcgactctagaggatccggccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatctctcgagtccggtactgttggtaaaatggaagacgccaaaaacataaagaaaggcccggcgccattctatcctctagaggatggaaccgctggagagcaactgcataaggctatgaagag

SEQ ID NO: 10 - DNA, 24 bp, S. Cerevisiae
  TL-lvc promoter
cgacggagtactgtcctccgagct

SEQ ID NO: 11 - DNA, 22 bp, Artificial Sequence
  hsp EcRe
tggacaagtgcattgaaccctt

SEQ ID NO: 12 - DNA, 26 bp, Artificial Sequence
  TK-luc reporter
gctaccaggtcaaaggtcacgtagct

SEQ ID NO: 13 - DNA, 26 bp, Artificial Sequence
  TK-luc reporter
gctaccaggtcaaaggtcacgtagct

SEQ ID NO: 14 - DNA, 27 bp, Artificial Sequence
  TIC-lvc repater
ccttaaggtgaataaccttggggctcc